(19) United States
(12) Patent Application Publication
(10) Pub. No.: US 2005/0106139 A1
(43) Pub. Date: May 19, 2005

(54) Humanized tissue factor antibodies

(76) Inventors: Ivan Svendsen, Smoerum (DK); Kristian Kjaergaard, Ballerup (DK); Stefan Zahn, Ballerup (DK)

Correspondence Address: NOVO NORDISK, INC.;PATENT DEPARTMENT, 100 COLLEGE ROAD WEST, PRINCETON, NJ, 08540, US

(21) Appl. No.: 10/699,362

(22) Filed: Oct. 31, 2003

Related U.S. Application Data
(60) Provisional application No. 60/427,157, filed on Nov. 18, 2002.

(30) Foreign Application Priority Data
Oct. 31, 2002 (DK) PA 2002 01661

(51) Int. Cl. A61K 39/395; C07K 16/44

(52) U.S. Cl. 424/141.1; 530/388.15

(57) ABSTRACT

Provided are novel isolated humanized antibodies that immunoreact with human tissue factor (TF) so as to inhibit the binding of coagulation factor Vila (FVIIa), pharmaceutical compositions comprising the same, methods of making the same, cells producing such antibodies, and methods of treating disease comprising administering such antibodies and/or compositions to a subject in need thereof. General methods for preparing humanized antibodies also are provided.

### CROSS-REFERENCE TO RELATED APPLICATIONS

This application claims priority under 35 U.S.C. 119 of Danish application no. PA 2002 01661 filed Oct. 31, 2002 and U.S. application No. 60/427,157 filed Nov. 18, 2002, the contents of which are fully incorporated herein by reference.

### FIELD OF THE INVENTION

The invention described herein relates to isolated antibodies that immunoreact with tissue factor (TF) to inhibit the binding of coagulation factor VIIa (FVIIa). The invention also relates to novel immunotherapeutic methods comprising administering humanized antibodies against TF to a subject to inhibit thrombus formation associated with surgery, microsurgery, angioplasty, or trauma or to inhibit thrombus formation and other functions of TF in abnormal haemostatic conditions associated with a disease (such as deep vein thrombosis, disseminated intravascular coagulation (DIC), coronary artery disease, sepsis, inflammation, atherosclerosis, or cancer). The invention also provides methods of preparing antibodies and novel cell lines for preparation of the humanized antibodies (abs).

### BACKGROUND OF THE INVENTION

Blood coagulation is a process consisting of a complex interaction of various blood components, or factors, which eventually gives rise to a fibrin clot. Generally, the blood components which participate in what has been referred to as the coagulation “cascade” are proenzymes or zymogens, enzymatically inactive proteins which are converted to proteolytic enzymes by the action of an activator, itself an activated clotting factor. Coagulation factors that have undergone such a conversion and generally referred to as “active factors,” and are designated by the addition of a lower case “a” suffix (e.g., Factor VIIa).

Activated Factor X (“Xa”) is required to convert prothrombin to thrombin, which then converts fibrinogen to fibrin as a final stage in forming a fibrin clot. There are two systems, or pathways, that promote the activation of Factor X. The “intrinsic pathway” refers to those reactions that lead to thrombin formation through utilization of factors present only in plasma. A series of protease-mediated activations ultimately generates Factor IXa which, in conjunction with Factor VIIIa, cleaves Factor X into Xa. An identical proteolysis is affected by FVIIa and its co-factor, TF, in the “extrinsic pathway” of blood coagulation. TF is a membrane bound protein and does not normally circulate in an active form in plasma. Upon vessel disruption, however, TF can complex with FVIIa to catalyze Factor X activation or Factor IX activation in the presence of Ca²⁺ and phospholipid. While the relative importance of the two coagulation pathways in hemostasis is unclear, Factor VII and TF have been found to play a pivotal role in the initiation of blood coagulation. For example, International Patent Application WO 92/15686 discloses the use of inactivated Factor VIIa for inhibiting blood coagulation.

It is often necessary to selectively block the coagulation cascade in a patient. Anticoagulants such as heparin, coumarin, derivatives of coumarin, indandione derivatives, or other agents may be used, for example, during kidney dialysis, or to treat deep vein thrombosis, disseminated intravascular coagulation (DIC), and other medical disorders. For example, heparin treatment or extracorporeal treatment with citrate ion may be used in dialysis to prevent coagulation during the course of treatment. Heparin is also used in preventing deep vein thrombosis in patients undergoing surgery.

Treatment with heparin and other anticoagulants may, however, have undesirable side effects. Available anticoagulants generally act throughout the body, rather than acting specifically at site of injury. Heparin, for example, may cause heavy bleeding. Furthermore, with a half-life of approximately 80 minutes, heparin is rapidly cleared from the blood, necessitating frequent administration. Because heparin acts as a cofactor for antithrombin III (ATIII), and ATIII is rapidly depleted in DIC treatment, it is often difficult to maintain the proper heparin dosage, necessitating continuous monitoring of ATIII and heparin levels. Heparin is also ineffective if ATIII depletion is extreme. Further, prolonged use of heparin may also increase platelet aggregation and reduce platelet count, and has been implicated in the development of heparin-induced thrombocytopenia. Indandione derivatives may also have toxic side effects. Several naturally occurring proteins also have been found to have some anticoagulant activity. ATIII also has been proposed as a therapeutic anticoagulant.

Antibodies against TF are known in the art. For example, therapeutic uses of mouse monoclonal antibodies (Mabs) against TF are disclosed in U.S. Pat. Nos. 6,001,978 and 5,223,427. International Patent Application WO 99/51743 discloses human/mouse chimera monoclonal antibodies directed against human TF. European Patent Application No. 833911 discloses CDR-grafted antibodies against human TF. Presta L. et al., Thrombosis and Haemostasis, Vol. 85 (3) pp. 379-389 (2001) describes humanized antibody against TF. Although these antibodies against TF may inhibit plasma clotting and TF/FVIIa-mediated activation of Factors IX and X, humanized antibodies that effectively immunoreact with tissue factor (TF) so as to inhibit the binding of coagulation factor VIIa (FVIIa) have yet to be described in the art.

In view of the foregoing, there remains a need for alternative and improved antibodies and compositions having anticoagulant activity, particularly for compositions which can be administered at relatively low doses and that do not produce the undesirable side effects associated with traditional anticoagulant compositions. The present invention fulfils this need by providing anticoagulants that act specifically at sites of injury, are free from such side effects, and further provide other related advantages disclosed herein. Furthermore the present invention provides compounds, which acts to inhibit the cellular functions of TF, which are implicated in conditions such as sepsis, inflammation, atherosclerosis, restenosis, and cancer. These and other advantages of the invention, as well as additional inventive features, will be apparent from the description of the invention provided herein.

### SUMMARY OF THE INVENTION

The invention described herein provides novel and useful antibodies that effectively immunoreact with TF so as to modulate the binding of FVIIa. In one exemplary aspect, the invention provides non-immunogenic high affinity humanized antibodies against human TF, which antibodies inhibit the binding of coagulation factor VII/VIIa. In another exemplary aspect, the invention provides novel and useful methods for selection of such therapeutically effective humanized antibodies against human TF. In a more particular exemplary aspect, the present invention relates to an isolated humanized antibody, which immunoreacts with a first epitope on human TF and inhibits the binding of human coagulation factor VIIa to human TF. In other aspects, the invention relates to methods of using these and other TF antibodies, methods of producing such antibodies, cells for producing such antibodies, and related compositions. In another exemplary aspect, the invention provides a novel method of producing humanized antibodies against proteins other than TF.

### BRIEF DESCRIPTION OF THE DRAWINGS

FIG. 1 is a schematic presentation of an exemplified screening assay for selection of human monoclonal high affinity antibodies against TF.

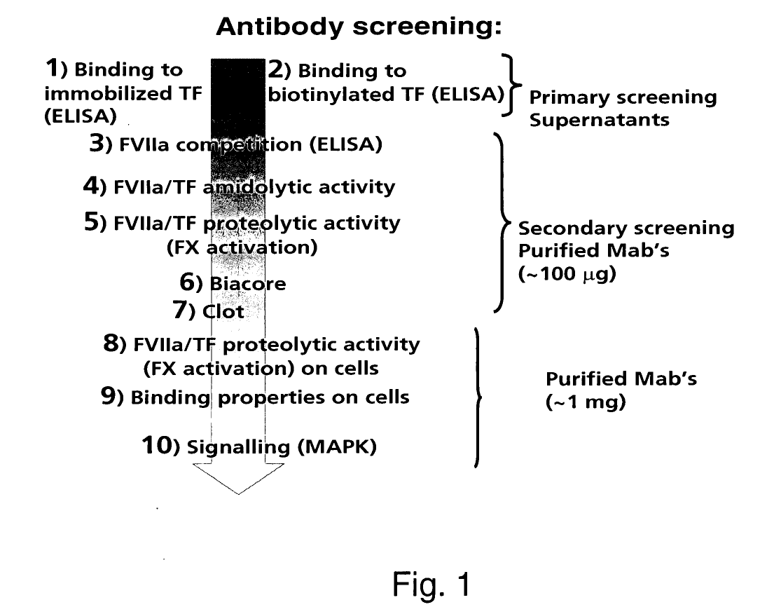

FIG. 2 is a detailed schematic representation of screening assays 1-3 as described in Example 1.

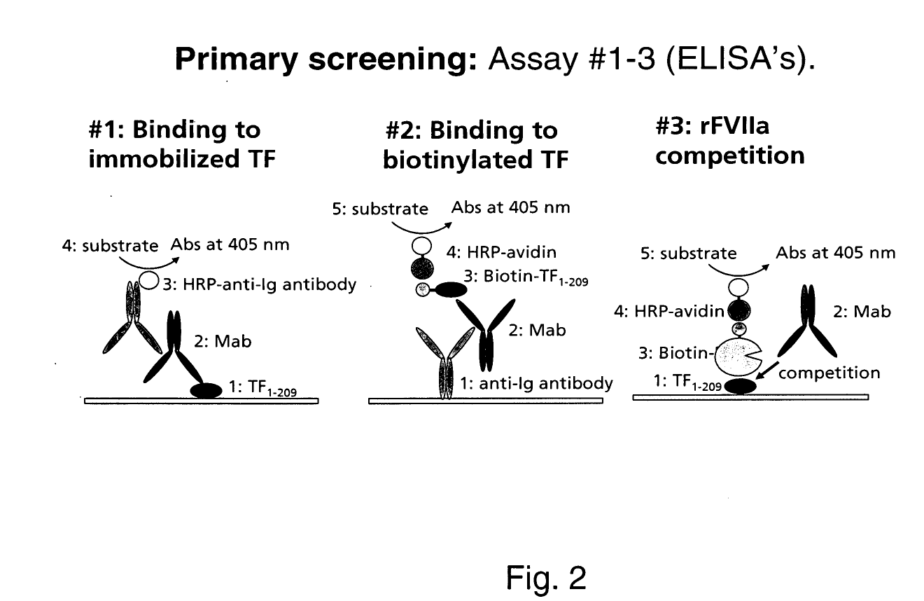

FIG. 3 is a detailed schematic representation of screening assays 4-7 as described in Example 1.

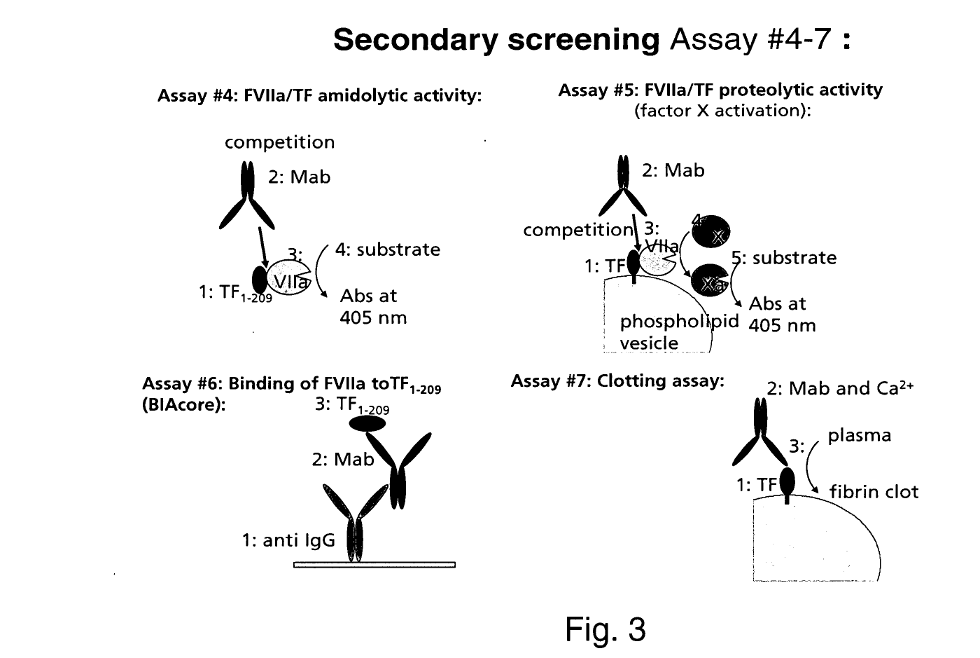

FIG. 4 is a detailed schematic representation of screening assays 8-10 as described in Example 1.

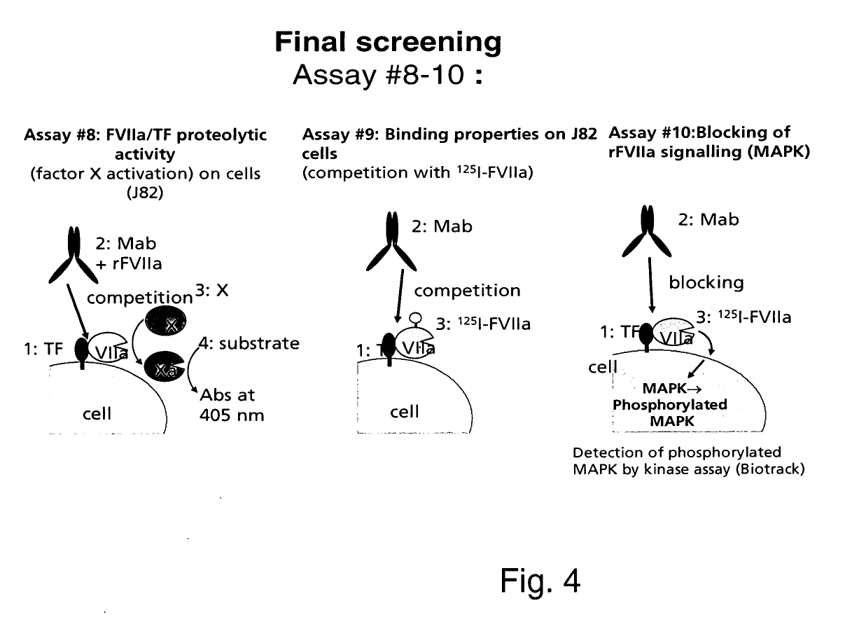

FIG. 5 provides an example of screening antibodies by assay no. 4, and shows inhibition of sTF/FVIIa amidolytic activity by FFR-rFVIIa (closed circles) and the human anti TF monoclonal antibody HuTF-31F2 (open circles).

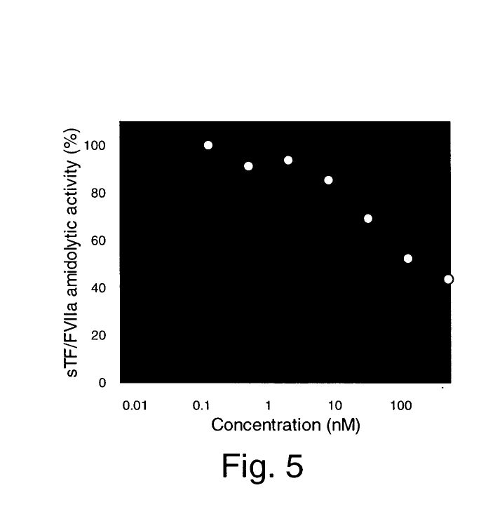

FIG. 6 illustrates an example of screening antibodies by assay no. 5 and shows inhibition of factor Xa generation by FFR-rFVIIa (closed circles) and the human anti-TF monoclonal antibody HuTF-31F2 (open circles).

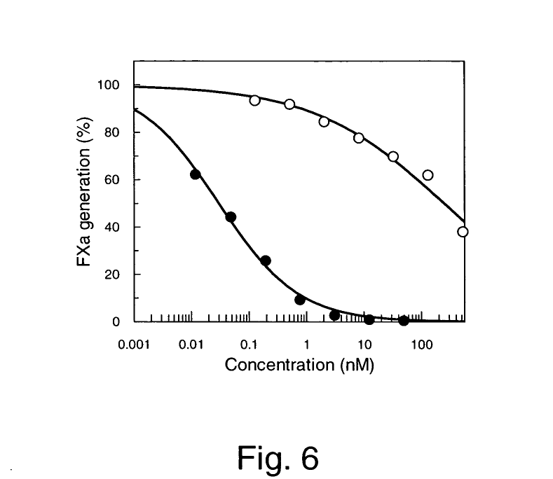

FIG. 7 depicts an example of screening antibodies by assay no. 7 and shows inhibition of human TF-induced clotting by FFR-rFVIIa (closed circles) and the human anti TF monoclonal antibody HuTF-31F2 (open circles).

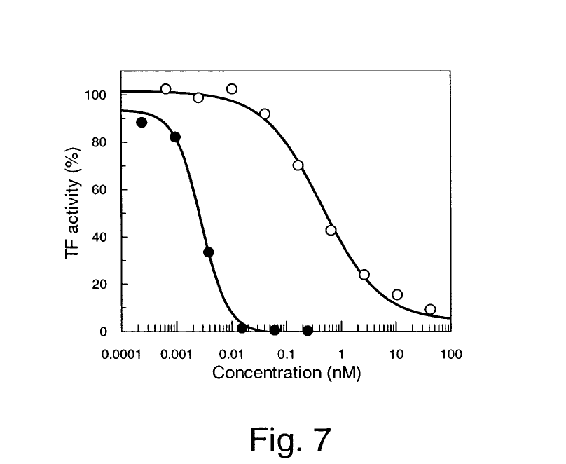

FIG. 8 provides an example of screening antibodies by assay no. 10. This Figure illustrates that only anti-TF monoclonal antibodies preventing FVIIa binding inhibit TF/FVIIa-mediated signaling.

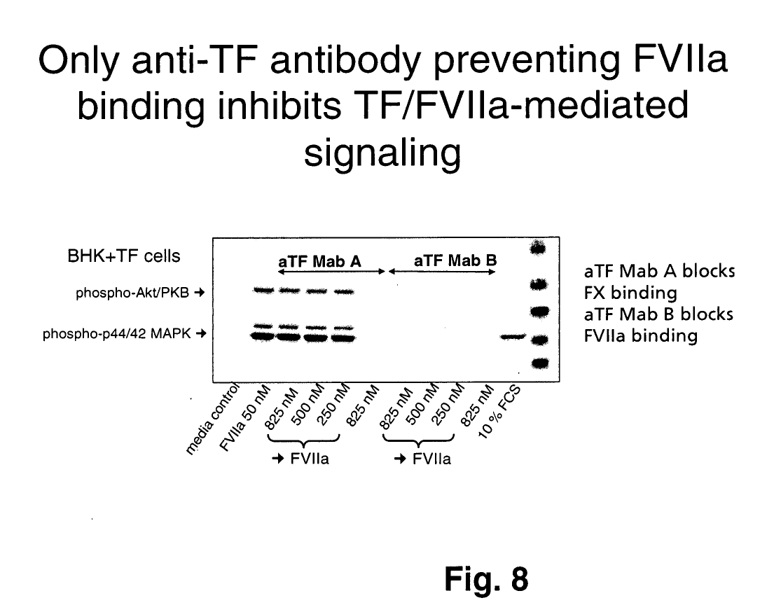

FIG. 9 illustrates human anti TF Mab inhibit FVIIa induced phosphorylation of p44/42 MAPK (assay no. 10). BHK cell transfected with TF were serum-starved for 2 hr to make cells quiescent. The antibodies HuMab 30F5 (500 nM) and HuMab 31F2 (500 nM) were added to the cells 15 min prior addition of FVIIa (15 nM). Cells were lysed and proteins were separate on SDS-PAGE and transferred to nitrocellulose by electroblotting. Western blot analysis was performed using polyclonal phospho-specific antibodies to p44/42 MAPK. Secondary antibodies were anti-rabbit IgG conjugated to Horse Radish Peroxidase. Detection of chemiluminescence was performed using a cooled CCD-camera. The bands on the digitalized picture were quantified and the band obtained with FVIIa was set to 100%. When cells were pre-incubated with HuMab 30F5 (500 nM) a 50% reduction in the phosphorylated band was observed and when cells were pre-incubated with HuMab 31F2 (500 nM) a 25% reduction was observed. In conclusion, this experiment show that the human antibodies against TF (30F5 and 31F2) partially inhibited the FVIIa induced phosphorylation of p44/42 MAPK. Similar results were obtained using 50 nM FVIIa.

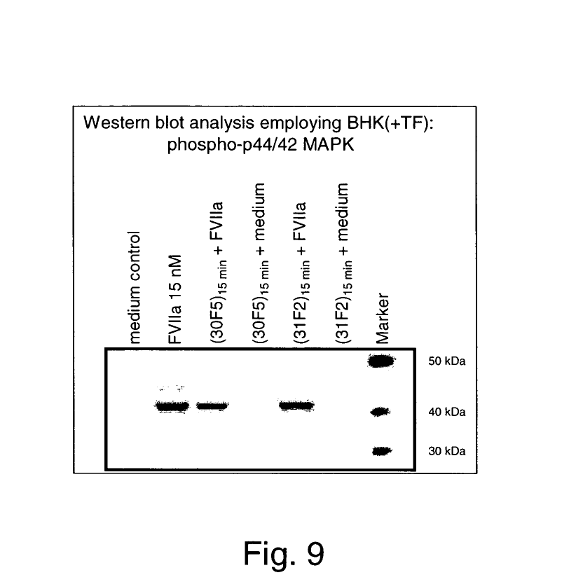

FIG. 10 illustrates an example of screening antibodies by assay no. 16. The figure demonstrates the inhibition of TF intracellular activity in TF expressing cells by monoclonal antibodies against TF. Anti TF Mab B inhibits TF intracellular activity, while Anti-TF Mab A do not.

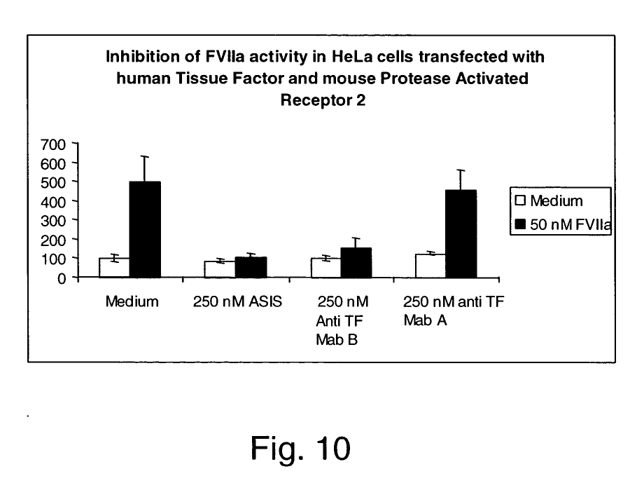

FIG. 11 illustrates an example of screening antibodies by assay no. 12. Specifically shown is a velocity profile of thromboelastograms obtained with 0.5 nM of FFR-rFVIIa and the human anti-TF antibody HuTF-31F2.

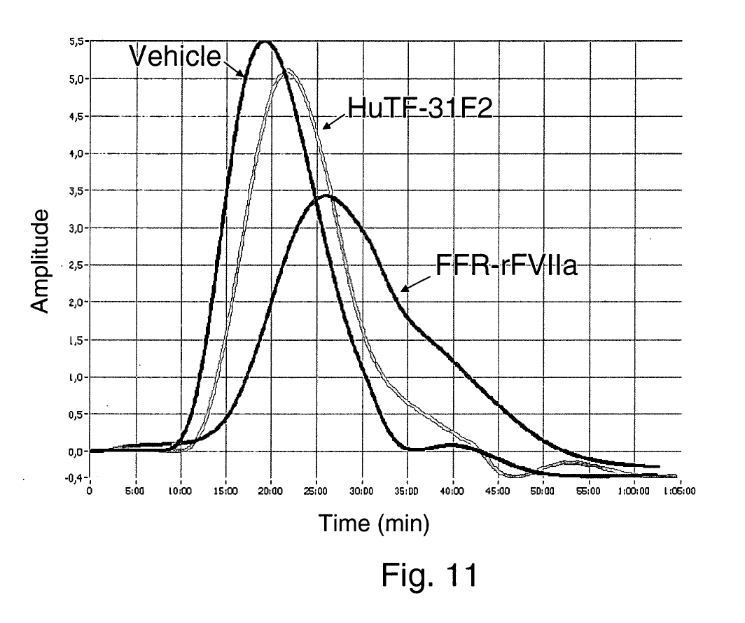

### DETAILED DESCRIPTION OF THE INVENTION

The terms “humanized antibodies” and “humanized antibody”, as used herein, refer to recombinant antibodies in which complementarity determining region (CDR) sequences or variants thereof derived from the germ line of another mammalian species, such as a mouse, have been grafted into (or otherwise combined with) human framework sequences. In one aspect of the invention, the invention provides an isolated humanized monoclonal antibody that comprises or has CDR amino acid sequences derived from a monoclonal antibody. In a further aspect of the invention, the invention provides an isolated humanized monoclonal antibody that comprises or has CDR amino acid sequences derived from a mouse monoclonal antibody.

The term “monoclonal antibody” as used herein, refers to a homogeneous population of immunoglobulins, i.e., the individual molecules of the antibody population are identical except for naturally occurring mutations. Antibodies are normally synthesized by lymphoid cells derived from B lymphocytes of bone marrow. Lymphocytes derived from the same clone produce immunoglobulin of a single amino acid sequence. Lymphocytes can not be directly cultured over long periods of time to produce substantial amounts of their specific antibody. However, Kohler et al., 1975, Nature, 256:495, demonstrated that a process of somatic cell fusion, specifically between a lymphocyte and a myeloma cell, could yield hybridoma cells which grow in culture and produce a specific antibody called a “monoclonal antibody”. Myeloma cells are lymphocyte tumor cells which, depending upon the cell strain, frequently produce an antibody themselves, although “non-producing” strains are known.

The term “recombinant antibody”, as used herein, is intended to include all antibodies that are prepared, expressed, created, or isolated by recombinant means, such as antibodies expressed using a recombinant expression vector transfected into a host cell (described further in Section II, below), antibodies isolated from a recombinant combinatorial antibody library (described further in Section III, below), antibodies isolated from an animal (e.g., a mouse) that is transgenic for human immunoglobulin genes (see e.g., Taylor, L. D., et al. (1992) Nucl. Acids Res. 20:6287-6295), and/or antibodies prepared, expressed, created or isolated by any other means that involves splicing of human immunoglobulin gene sequences to other DNA sequences. Such recombinant human antibodies have variable and constant regions derived from human germ line immunoglobulin sequences. In certain aspects of the invention, however, such recombinant human antibodies are subjected to in vitro mutagenesis (or, when an animal transgenic for human Ig sequences is used, in vivo somatic mutagenesis) and, thus, the amino acid sequences of the VH and VL regions of the recombinant antibodies may be sequences that, while derived from and related to human germ line VH and VL sequences, may not naturally exist within the human antibody germ line repertoire in vivo.

The terms “human tissue factor” or “human TF” as used herein, refers to the full length polypeptide receptor comprising the amino acid sequence 1-263 of native human tissue factor.

The term “antibody”, as used herein, refers to immunoglobulin molecules and fragments thereof, which have the ability to specifically bind to an antigen (e.g., human TF). Full-length antibodies comprise four polypeptide chains, two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2, and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. Thus, included within the definition of an antibody are fragments of an antibody that retain the ability to specifically bind to an antigen (e.g., human TF). It has been shown that the antigen-binding function of an antibody can be performed by fragments of a full-length antibody. Examples of binding fragments encompassed within the term “antibody” include (i) a Fab fragment, a monovalent fragment consisting of the VL, VH, CL, and CH1 domains; (ii) F(ab)₂ and F(ab′)₂ fragments, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody; (v) a dAb fragment (see, e.g., Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426: and Huston et al. (1988) Proc. Natl. Acad. Sci. USA 85:5879-5883). Such single chain antibodies are also intended to be encompassed within the term “antibody”. Other forms of single chain antibodies, such as diabodies are also encompassed by the term “antibody.” Diabodies are bivalent and bispecific antibodies in which VH and VL domains are expressed on a single polypeptide chain, but are separated by a linker that is too short to allow for pairing between the two domains on the same chain, thereby forcing the domains to pair with complementary domains of another chain so as to create two antigen binding sites (see e.g., Holliger, P., et al. (1993) Proc. Natl. Acad. Sci. USA 90:6444-6448; Poljak, R. J., et al. (1994) Structure 2:1121-1123). It is understood that human TF may have one or more antigenic determinants comprising (1) peptide antigenic determinants which consist of single peptide chains within human TF; (2) conformational antigenic determinants which consist of more than one spatially contiguous peptide chains whose respective amino acid sequences are located disjointedly along the human TF polypeptide sequence; and (3) post-translational antigenic determinants which consist, either in whole or part, of molecular structures covalently attached to human TF after translation, such as carbohydrate groups.

An “isolated humanized antibody”, as used herein, refers to a humanized antibody that is substantially free of other antibodies having different antigenic specificities (e.g., an isolated antibody that specifically binds human TF is substantially free of antibodies that specifically bind antigens other than human TF). An isolated antibody that specifically binds human TF may, however, have cross-reactivity to other antigens, such as TF molecules from other species (discussed in further detail below). Moreover, an isolated antibody may be substantially free of other cellular material and/or chemicals.

The term “epitope” as used herein means any antigenic determinant on an antigen to which the antibody binds. Epitopic determinants usually consist of chemically active surface groupings of molecules such as amino acids or sugar side chains and usually have specific three dimensional structural characteristics, as well as specific charge characteristics.

The terms “immunoreacts” or “immunoreacting”, as used herein, means any binding of an antibody to its epitope with a dissociation constant K_{d} lower than 10⁻⁴ M. The terms “immunoreacts” or “immunoreacting” are used where appropriate interchangeably with the phrase “specifically bind”.

The term “inhibits”, as used herein, means any reduction compared to a reference. As an example, an antibody that “inhibits” the binding of human coagulation factor Vila to human TF means any antibody that reduces the ability of human coagulation factor VIIa to bind human TF compared to the ability of human coagulation factor VIIa to bind human TF in the absence of the antibody.

The term “affinity”, as used herein, means the strength of the binding of an antibody to an epitope. The affinity of an antibody is measured by the dissociation constant K_{d}, defined as [Ab]×[Ag]/[Ab−Ag] where [Ab−Ag] is the molar concentration of the antibody-antigen complex, [Ab] is the molar concentration of the unbound antibody and [Ag] is the molar concentration of the unbound antigen. The affinity constant Kₐ is defined by 1/K_{d}. Preferred methods for determining antibody specificity and affinity by competitive inhibition can be found in Harlow, et al., Antibodies: A Laboratory Manual, Cold Spring Harbor Laboratory Press, Cold Spring Harbor, N.Y., (1988), Colligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference.

In another aspect, the invention relates to a pharmaceutical composition comprising a therapeutically effective amount of a humanized antibody, which immunoreacts with a first epitope (also referred to as “epitope A”) on human TF and inhibits the binding of human coagulation factor VIIa to human TF.

The phrase “a therapeutically effective amount” refers to an amount sufficient to promote, induce, and/or enhance treatment or other therapeutic effect in a subject. A “physiologically effective amount” refers to an amount of antibody or antibody-comprising composition sufficient to detectably inhibit TF binding to FVIIa in a subject (e.g., throughout the subject or in one or more tissues wherein such inhibition is sought).

Typically, methods of the invention that involve administration of antibodies or antibody-comprising compositions can be practiced using any suitable dose of the antibodies or composition. A particular dose of composition to be administered to a subject will typically be determined by a qualified practitioner, who may titrate dosages to achieve the desired response, using standard techniques and/or relatively routine experimentation. Factors for consideration of dose can include potency, bioavailability, desired pharmacokinetic and/or pharmacodymamic profiles, condition of treatment (e.g. trauma, inflammation, septic chock), patient-related factors (e.g. weight, health, age, etc.), presence of co-administered medications, time of administration, and/or other factors known to a typical medical practitioner. The dosage of a humanized antibody against TF administered to a patient may also vary with the type and severity of the condition to be treated, but is generally in the range of 0.1-5.0 mg/kg body weight.

The phrases “framework amino acid sequence” and “framework sequence” individually refer to an amino acid sequence of an antibody that is not a CDR sequence. A framework sequence that is derived from a parent antibody consists of the amino acids present in the corresponding non-CDR sequence of the parent antibody from which such sequences are considered to be derived. Unless otherwise stated or clearly contradicted by context, any framework sequence incorporated in any antibody described herein can be substituted with a suitable framework sequence variant. A framework sequence variant can be any sequence of amino acids that (a) has similar biological property as an antibody produced by a mammal in response to an antigen of interest (e.g., permits immunogenic binding of the antibody of which it is a part to its target, such as TF, preferably with an affinity that is at least about as great, if not greater, than its mammalian-produced counterpart and desirably allows the antibody to retain a similar biological effect as its mammalian-produced counterpart—e.g., a similar level of inhibition of TF binding to FVIIa) and (b) has a high level of amino acid sequence identity with its mammalian-produced counterpart (e.g., has at least about 80%, at least about 85%, at least about 90%, or at least about 95% identity to). Typically, a framework sequence variant will vary by only a few amino acids from its mammalian-produced counterpart (e.g., a variant may differ by 3, 2, or 1 substitutions, deletions, and/or additions) and/or will vary only in terms of conservative amino acid substitutions. Often, a framework sequence variant will consist essentially of the same sequence as the framework sequence of its mammalian-produced counterpart.

The term “subject” as used herein is intended to mean any animal, in particular mammals, such as humans, and may, where appropriate, be used interchangeably with the term “patient”.

In another aspect, the invention relates to a composition comprising a humanized antibody, which immunoreacts with an epitope A present on human TF and inhibits the binding of human coagulation factor VIIa to human TF.

In a further aspect, the invention relates to a method for treatment of a FVIIa/TF related disorder in a human, which method comprises administering to the human a therapeutically effective amount of a humanized antibody that immunoreacts with an epitope A present on human TF and inhibits the binding of human coagulation factor VIIa to human TF.

“Treatment” means the administration of an amount of antibodies of the invention and/or a compound of the invention with the effect of reducing the intensity of, delaying the onset of, and/or reducing the risk of, and preferably preventing, the occurrence of one or more physiological symptoms associated with a disease state or reducing and/or eliminating one or more detectable physiological conditions or symptoms associated with a disease state present in a subject (and, preferably, eliminating all conditions associated with the disease state). Humanized TF antibodies and compositions provided by the invention can be used in any and all aspects of treating a subject.

The term “FVIIa/TF related disorder” as used herein means a disease or disorder, where TF and FVIIa are involved. Typically, a FVIIa/TF related disorder is a disease where TF signaling is detectably modulated as compared to such signaling in a typically healthy individual. For example, in some FVIIa/TF related disorders TF signaling will be substantially impaired as compared to such signaling in a healthy individual. Examples of FVIIa/TF disorders include thrombotic and/or coagulopathic related diseases, disorders including inflammatory response and chronic thromboembolic diseases, and disorders associated with fibrin formation, including vascular disorders such as deep venous thrombosis, arterial thrombosis, post surgical thrombosis, coronary artery bypass graft (CABG), percutaneous transdermal coronary angioplastry (PTCA), stroke, tumor growth, tumor metastasis, angiogenesis-related disorders, thrombolysis, atherosclerosis, arteriosclerosis (e.g., arteriosclerosis following angioplastry), and restenosis (such as restenosis following angioplastry), acute and chronic indications such as inflammation, septic chock, septicemia, hypotension, adult respiratory distress syndrome (ARDS), disseminated intravascular coagulopathy (DIC), pulmonary embolism, platelet deposition, myocardial infarction, or the prophylactic treatment of mammals with atherosclerotic vessels at risk for thrombosis, and other diseases or disorders. The FVIIa/TF related disorder is not limited to in vivo coagulopatic disorders such as those named above, but also includes ex vivo FVIIa/TF-related processes such as coagulation that may result from the extracorporeal circulation of blood (including blood removed in-line from a patient in such processes as dialysis procedures, blood filtration, or blood bypass during surgery).

The term “Factor VIIa”, or “FVIIa” means “Iwo chain” activated coagulation factor VII cleaved by specific cleavage at the Arg152-Ile153 peptide bond. FVIIa, may be purified from blood or produced by recombinant means. It is evident that the practice of the methods described herein is independent of how the purified factor VIIa is derived and, therefore, the applicable aspects of the invention can include the use of any suitable Factor VIIa preparation. In many aspects, human Factor FVIIa is preferred.

The term “FVII” means “single chain” coagulation factor VII.

In a further aspect, the invention relates to a method for preparing humanized antibodies. The method comprises preparing humanized antibodies against human TF and (a) testing the antibodies in a FVIIa/TF amidolytic assay and selecting a humanized antibody which inhibits TF-induced FVIIa amidolytic activity, with an IC₅₀ value lower than the IC₅₀ value of FFR-rFVIIa+100 nM (using 10 nM FVIIa in the assay) (such as lower than the IC₅₀ value of FFR-rFVIIa+40 nM, preferably lower than the IC₅₀ value of FFR-rFVIIa+20 nM, more preferably lower than the IC₅₀ value of FFR-rFVIIa+10 nM, and even more preferably lower than the IC₅₀ value of FFR-rFVIIa, (b) testing the antibodies in a FVIIa competition assay and selecting humanized antibody which compete with FVIIa binding, or (c) testing antibodies in a FVIIa signaling assay and selecting humanized antibodies which inhibit FVIIa-induced intracellular signaling.

The term “FVIIa signaling assay” as used herein means any assay which measures the FVIIa-induced intracellular signaling.

The term “FVIIa-induced intracellular signaling” as used herein means the intracellular events mediated when FVIIa is added to the a human cell. “FVIIa-induced intracellular signaling” may be measured by the MAPK signaling assay or a gene expression analysis assay which measures the up-regulation of genes independently selected from the list comprising Fra-1, Id2, and Cyr61.

Unless otherwise indicated, the specific IC₅₀ values referred to with respect to the TF-induced clot assays described herein are those obtained when using normal human plasma.

The term “TF-induced clot assay” as used herein means any assay where clotting time is measured in sample comprising blood plasma and TF. An example of a TF-induced clot assay is described in Example 1, assay 7.

The term “FXa generation assay” as used herein means any assay where activation of FX is measured in a sample comprising TF, FVIIa, FX, calcium, and phospholipids. An example of a FXa generation assay is described in Example 1, assay 5.

The term “FVIIa/TF amidolytic assay” as used herein means any assay where the amidolytic activity (i.e., the cleavage of a small peptide substrate) of FVIIa is measured in the presence of TF. An example of a FVIIa/TF amidolytic assay is described in Example 1, assay 4.

The term “TF ELISA assay” as used herein means any ELISA assay comprising TF and antibodies against TF. Examples of TF ELISA assays are the direct and indirect TF ELISA assays described in Example 1, assay 1 and 2.

The term “direct TF ELISA assay” as used herein means any TF ELISA assay comprising immobilized TF. An example of a direct TF ELISA assays is described in Example 1, assay 1.

The term “indirect TF ELISA assay” as used herein means any TF ELISA assay, where TF is in solution. An example of a direct TF ELISA assays is described in Example 1, assay 2.

In a further aspect, the invention relates to a humanized antibodies that immunoreact with an epitope A present on human TF and inhibits the binding of human coagulation factor VIIa to human TF and are produced by a method that comprises preparing humanized antibodies against human TF, and (a) testing the antibodies in a FVIIa/TF amidolytic assay and selecting a humanized antibody therefrom that inhibits TF-induced FVIIa amidolytic activity, with an IC₅₀ value lower than the IC₅₀ value of FFR-rFVIIa+100 nM (using 10 nM FVIIa) (such as lower than the IC₅₀ value of FFR-rFVIIa+40 nM, preferably lower than the IC₅₀ value of FFR-rFVIIa+20 nM, more preferably lower than the IC₅₀ value of FFR-rFVIIa+10 nM, and even more preferably lower than the IC₅₀ value of FFR-rFVIIa), or (b) testing the antibodies in a FVIIa competition assay and selecting humanized antibody therefrom that compete with FVIIa binding, or (c) testing the antibodies in a FVIIa signaling assay and selecting humanized antibodies therefrom that inhibit FVIIa-induced intracellular signaling.

In yet another aspect, the invention relates to a method for preparation of a humanized antibody that includes (i) preparing immortal cells that secrete humanized antibodies, (ii) isolating culture medium from the immortal cells comprising produced humanized antibodies, and (iii) testing the isolated antibodies in (a) an indirect TF ELISA assay comprising TF in solution and selecting humanized antibodies which immunoreacts with human TF in solution, (b) in a FVIIa competition assay and selecting humanized antibodies which competes with FVIIa binding, (c) in a FVIIa/TF amidolytic assay and selecting humanized antibody which inhibit TF-induced FVIIa amidolytic activity, with an IC₅₀ value lower than the IC₅₀ value of FFR-rFVIIa+100 nM (using 10 nm FVIIa in the assay), such as lower than the IC₅₀ value of FFR-rFVIIa+40 nM, preferably lower than the IC₅₀ value of FFR-rFVIIa+20 nM, preferably lower than the IC₅₀ value of FFR-rFVIIa+10 nM, and more preferably lower than the IC₅₀ value of FFR-rFVIIa, (d) in a FXa generation assay and selecting humanized antibody which inhibit FXa generation with an IC₅₀ value lower than the IC₅₀ value of FFR-rFVIIa+100 nM (using 0.1 nM FVIIa in the assay), such as lower than the IC₅₀ value of FFR-rFVIIa+10 nM preferably lower than the IC₅₀ value of FFR-rFVIIa+5 nM, preferably lower than the IC₅₀ value of FFR-rFVIIa+1 nM, more preferably lower than the IC₅₀ value of FFR-rFVIIa+0.1 nM, more preferably lower than the IC₅₀ value of FFR-rFVIIa, (e) in a TF-induced clot assay and selecting humanized antibodies which inhibits clot formation in this assay with an IC₅₀ value lower than the IC₅₀ value of FFR-rFVIIa+1 nM, such as lower than the IC₅₀ value of FFR-rFVIIa+500 pM, preferably lower than the IC₅₀ value of FFR-rFVIIa+200 pM, preferably lower than the IC₅₀ value of FFR-rFVIIa+100 pM, such as lower than the IC₅₀ value of FFR-rFVIIa+50 pM, preferably lower than the IC₅₀ value of FFR-rFVIIa+10 pM, more preferably lower than the IC₅₀ value of FFR-rFVIIa+5 pM, more preferably lower than the IC₅₀ value of FFR-rFVIIa, or (f) in any combination of the tests of (a)-(e) and selecting humanized antibodies that meet the criteria set forth above with respect to these tests, and (iv) selecting and cultivating an immortal cell producing the selected humanized antibody (or at least cultivating an immortal cell producing the selected humanized antibody), and (v) isolating the selected antibody (optionally, the method can further include preparing a pharmaceutically acceptable composition comprising the selected antibody). In a further aspect, the invention relates to a humanized antibody which immunoreacts with an epitope A present on human TF, inhibits the binding of human coagulation factor VIIa to human TF, and is produced by this method described above.

The term “antibody-producing cell” as used herein means any cell capable of producing an antibody. Antibody-producing cells include hybridomas, transfected cell lines, and the relatively short-lived, or mortal, splenocytes and lymphocytes from a mammal that have been injected with an antigen.

In a further aspect, the invention relates to a cell producing humanized antibodies that immunoreact with an epitope A present on human TF and inhibit the binding of human coagulation factor VIIa to human TF. In one aspect, the cell is a mammalian cell. In a further aspect, the cell is isolated from a mouse. In another aspect, the cell is a hybridoma. In a further aspect, the cell is a myeloma cell.

Many myeloma cell lines may be used for the production of humanized antibodies in accordance with the techniques described herein, including, e.g., Mouse myeloma NSO (ECACC 85110503), rat myeloma YB2/0 (ATCC CRL 1662), P3X63-Ag8, P3X63-AG8.653, P3/NS1-Ag4-1 (NS-1), Sp2/0-Agl4, and S194/5.XXO.Bu.1. The P3×63-Ag8 and NS-1 cell lines have been described by Kohler and Milstein (Eur. J. Immunol. 6:511 (1976)). Shulman et al. (Nature 276:269 (1978)) developed the Sp2/0-Ag14 myeloma line. The S194/5.XXO.Bu.1 line was reported by Trowbridge (J. Exp. Med. 148:313 (1979)).

Traditionally, humanized or human antibodies are developed by phage display, ribosome display, RNA display, surface display on a “living organism,” by using (e.g., expression in) transgenic animals, or by introducing human immune competent cells to immune incompetent animals. However, humanized antibodies produced by these techniques have affinities that are below what is normally needed for therapeutic antibodies. One has to “affinity mature” these humanized antibodies further. One reason for the low affinity of such antibodies is that CDR regions from large libraries are combined with a “naive” human framework that has never seen the epitope of interest before, which makes the conformation of this human framework sub-optimal. Using transgenic animals where the human Ig repertoire has been introduced to produce human antibodies will not always result in high affinity antibodies (probably due to a sub optimally functioning immune system of the transgenic animals). Immunizing immune compromised animals that have received human immune competent cells is another way of developing human antibodies; however, the length of the immunization strategy that can be used in this method often is not sufficient for obtaining high affinity antibodies.

In many aspects of this invention, fully human antibodies generated by one of the above mentioned animal systems are combined with CDR sequences from high affinity antibodies generated in another animal, wherein both the fully human antibody and the CDR sequences from high affinity antibodies generated in the other animal are directed against the same target protein.

Thus, in a further particular aspect, the invention relates to an isolated humanized antibody, which immunoreacts with an epitope P present on a protein, wherein the human framework amino acid sequences of said humanized antibody are derived from a human antibody that immunoreacts with an epitope H present on the protein, wherein epitope H comprises an amino acid residue at a particular position of the protein that also is comprised within epitope P. In such aspects of the invention, the protein can be any suitable protein. For example, the protein may be a protein associated with a particular biological effect and/or disease in a subject.

In another aspect, the CDR amino acid sequences of the isolated humanized antibody are derived from a parent monoclonal antibody. In one aspect, the parent monoclonal antibody is a mouse monoclonal antibody. In one aspect, the CDR amino acid sequences are variant CDR amino acid sequences relative to the CDR amino acid sequences of a parent monoclonal antibody. The term “variant”, as used herein with respect to CDR amino acid sequences, designates CDR amino acid sequences, wherein one or more amino acids have been substituted by another amino acid residue, one or more amino acids have been deleted, and/or one or more amino acids have been inserted into the CDR amino acid sequence.

A CDR amino acid sequence “variant” can differ from its most closely related wild-type CDR amino acid sequence counterpart by any suitable amount, so long as the variant exhibits CDR functionality (e.g., conference of antigen specificity to the antibody with which it is associated). For example, variant CDR amino acid sequences that share about 70% sequence identity to the CDR sequences of a parent monoclonal antibody can be suitable (e.g., at least about 75% identity). Typically, variant CDR amino acid sequence share at least about 80% sequence identity, at least about 90% sequence identity, or at least about 95% sequence identity with the CDR sequences of a parent monoclonal antibody. In other aspects, a variant CDR amino acid sequence can have two, three, or four amino acid substitutions relative to the CDR sequences of the parent monoclonal antibody. In such aspects, a CDR variant can consist essentially of a sequence derived from a mammalian antibody.

In another aspect, the invention provides useful and antigenic fragments of the humanized antibodies described herein. For example, in one aspect, the isolated humanized antibody is a Fab fragment. In another aspect, the isolated humanized antibody is a F(ab)₂ fragment. In a further aspect, the isolated humanized antibody is a F(ab′)₂ fragment. In yet another aspect, the isolated humanized antibody is a single chain Fv fragment.

In a further aspect, the isolated humanized antibody has human framework amino acid sequences derived from a human antibody, which parent antibody immunoreacts with a second epitope (or “epitope B”) present on human TF. In one aspect, epitope B comprises an amino acid residue at a particular position of human TF that is also comprised within epitope A with which said humanized antibody immunoreacts. In one aspect, epitope A comprises an amino acid residue at a particular position of human TF selected from the list consisting of Trp45, Lys46 and Tyr94. In a further aspect, epitope B comprises an amino acid residue at a particular position of human TF selected from the list consisting of Trp45, Lys46, and Tyr94.

The terms “human antibody”, “human antibodies”, “human TF antibody”, and “human TF antibodies”, as used herein, refer to antibodies having variable and constant regions derived from human germ line immunoglobulin sequences. The human antibodies of the invention may include amino acid residues not encoded by human germ line immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences are derived from the germ line of another mammalian species, such as a mouse, have been grafted onto human framework sequences, e.g. the so-called humanized antibodies.

In a further aspect of the invention, the isolated humanized antibody has a K_{d} for binding to human TF within the range of about 10⁻¹⁵-10⁻⁸ M. It is to be understood, that the K_{d} for humanized antibody binding to human TF referred to is as determined in an assay, wherein the humanized antibody is immobilized (see, e.g., assay 6 for an example of such an assay). In further aspects of the invention the isolated humanized antibody has a K_{d} for binding to human TF within the range of about 10⁻¹⁵-10⁻¹⁰ M or about 10⁻¹⁵-10⁻¹² M.

The antibodies of the invention can exhibit any suitable level of binding to human TF. In one exemplary aspect of the invention, the isolated humanized antibody has a K_{d} for binding to human TF of less than about 10⁻⁸ M. In another aspect of the invention the isolated humanized antibody has a K_{d} for binding to human TF of less than about 10⁻⁹ M. In other aspects, the isolated humanized antibody has a K_{d} for binding to human TF that is less than about 10⁻¹⁰ M, less than about 10⁻¹¹ M, less than about 10⁻¹² M, less than about 10⁻¹³ M, less than about 10⁻¹⁴ M, or less than about 10⁻¹⁵ M.

In a further aspect, the invention provides a method for preparing a humanized antibody that comprises testing antibodies in a TF-induced clot assay and selecting a humanized antibody which inhibit clot formation in this assay with an IC₅₀ value that is less than about 1 nM. For example, in a more particular aspect, the method can include selecting a humanized antibody that inhibits clot formation in the assay with an IC₅₀ value that is less than about 500 pM. The method also can be practiced such that antibodies are selected where the IC₅₀ value in the assay is less than about 200 pM, less than about 100 pM, less than about 50 pM, less than about 10 pM, or less than about 5 pM.

The invention also provides a method of preparing a humanized antibody that comprises testing antibodies in a TF-induced clot assay and selecting a humanized antibody which inhibit clot formation in this assay with an IC₅₀ value of less than the IC₅₀ value of FFR-rFVIIa+about 1 nM, such as less than the IC₅₀ value of FFR-rFVIIa+about 500 pM, less than the IC₅₀ value of FFR-rFVIIa+about 200 pM, less than the IC₅₀ value of FFR-rFVIIa+about 100 pM, less than the IC₅₀ value of FFR-rFVIIa+about 50 pM, less than the IC₅₀ value of FFR-rFVIIa+about 10 pM, less than the IC₅₀ value of FFR-rFVIIa+about 5 pM, or less than the IC₅₀ value of FFR-rFVIIa.

In a further aspect of the invention the method for preparing a humanized antibody that comprises testing antibodies in a FXa generation assay and selecting a humanized antibody which inhibit FXa generation with an IC₅₀ value less than about 100 nM (in an assay with a FVIIa concentration of 0.1 nM). In other aspects, the selected antibody inhibit FXa generation with an IC₅₀ value that is less than about 10 nM, less than about 5 nM, less than about 1 nM, or less than about 0.1 nM.

The invention also provides a method of preparing a humanized antibody that comprises testing antibodies in a FXa generation assay and selecting a humanized antibody which inhibit FXa generation with an IC₅₀ value that is less than the IC₅₀ value of FFR-rFVIIa+about 100 nM (using 0.1 nM FVIIa in the assay), such as less than the IC₅₀ value of FFR-rFVIIa+about 10 nM, less than the IC₅₀ value of FFR-rFVIIa+about 5 nM, less than the IC₅₀ value of FFR-rFVIIa+about 1 nM, less than the IC₅₀ value of FFR-rFVIIa+about 0.1 nM, or less than the IC₅₀ value of FFR-rFVIIa.

The invention further provides a method of preparing a humanized antibody that comprises testing antibodies in a FVIIa/TF amidolytic assay and selecting a humanized antibody which inhibits TF-induced FVIIa amidolytic activity with an IC₅₀ value of less than about 100 nM (in an assay with a FVIIa concentration of 10 nM). In further aspects, the antibody inhibits TF-induced FVIIa amidolytic activity with an IC₅₀ value of less than about 40 nM, less than about 20 nM, less than about 10 nM, or less than about 5 nM.

The invention additionally provides a method of preparing a humanized antibody that comprises testing antibodies in a FVIIa/TF amidolytic assay and selecting a humanized antibody which inhibits TF-induced FVIIa amidolytic activity, with an IC₅₀ value of less than the IC₅₀ value of FFR-rFVIIa+about 100 nM (using 10 nM FVIIa in the assay), such as less than the IC₅₀ value of FFR-rFVIIa+about 40 nM, less than the IC₅₀ value of FFR-rFVIIa+about 20 nM, less than the IC₅₀ value of FFR-rFVIIa+about 10 nM, or less than the IC₅₀ value of FFR-rFVIIa.

In another aspect of the invention, preparing humanized antibodies by one of the techniques described herein comprises testing antibodies in a FVIIa competition assay and selecting a humanized antibody which detectably competes with FVIIa binding.

In yet other aspects of the invention, preparing a humanized antibody by one of the methods described herein further comprises testing antibodies in a TF ELISA assay comprising TF and selecting a humanized antibody which detectably binds human TF.

In still other aspects of the invention, a method of preparing a humanized antibody by one of the methods described herein further comprises testing antibodies in a direct TF ELISA assay comprising immobilized TF and selecting humanized antibodies which bind immobilized human TF.

In a further aspect of the invention, preparing a humanized antibody by one of the techniques described herein comprises testing antibodies in an indirect TF ELISA assay comprising TF in solution and selecting humanized antibodies which bind human TF in solution.

The invention also provides a method for preparing a humanized antibody that comprises testing antibodies in a FXa generation assay on TF expressing cells and selecting humanized antibodies which inhibit FXa generation on TF expressing cell with an IC₅₀ value of less than about 500 nM (in an assay with a FVIIa concentration of 1 nM). In further aspects, the method comprises selecting antibodies that inhibit FXa generation with an IC₅₀ value of less than about 100 nM, less than about 50 nM, less than about 10 nM, or less than about 5 nM.

In another aspect, the invention provides a method for preparing a humanized antibody that comprises testing antibodies in a FXa generation assay on TF expressing cells and selecting humanized antibodies which inhibit FXa generation on TF expressing cell with an IC₅₀ value of less than the IC₅₀ value of FFR-rFVIIa+about 500 nM (using 1 nM FVIIa in the assay), such as less than the IC₅₀ value of FFR-rFVIIa+about 100 nM, less than the IC₅₀ value of FFR-rFVIIa+about 50 nM, less than the IC₅₀ value of FFR-rFVIIa+about 10 nM, less than the IC₅₀ value of FFR-rFVIIa+about 5 nM, or less than the IC₅₀ value of FFR-rFVIIa. The term “TF expressing cell” means any mammalian cell that expresses human TF.

In another aspect, the invention provides a method of preparing a humanized antibody that comprises testing antibodies in a whole cell TF binding assay and selecting humanized antibodies which competes with FVIIa binding to human TF expressed on the cell surface of whole cells.

The invention also provides a method for preparing a humanized antibody that comprises testing antibodies in a biosensor assay and selecting humanized antibodies with a K_{d} value for binding to human TF of less than about 100 nM. In further aspects, the K_{d} value for binding to human TF is less than about 10 nM, less than about 5 nM, less than about 1 nM, or less than about 0.5 nM. In a further aspect the K_{d} value for binding to human TF is less than about 10⁻¹⁰ M, less than about 10⁻¹¹ M, less than about 10⁻¹² M, less than about 10⁻¹³ M, less than about 10⁻¹⁴ M, or less than about 10⁻¹⁵ M.

In a further aspect of the invention the preparation of a humanized antibody comprises testing antibodies in a MAPK signaling assay and selecting humanized antibodies which inhibit FVIIa-induced activation of the MAPK signaling. In one aspect, the humanized antibody inhibits FVIIa-induced activation of the MAPK signaling with 98% efficiency (wherein about 100% efficiency equals the complete inhibition of MAPK signaling within the means of detection). In one aspect, the humanized antibody inhibits FVIIa-induced activation of the MAPK signaling with about 90% efficiency. In one aspect the humanized antibody inhibits FVIIa-induced activation of the MAPK signaling with about 70% efficiency. In one aspect the humanized antibody inhibits FVIIa-induced activation of the MAPK signaling with about 50% efficiency. In one aspect the humanized antibody inhibits FVIIa-induced activation of the MAPK signaling with about 30%.

The term “MAPK signaling” means a cascade of intracellular events that mediate activation of Mitogen-Activated-Protein-Kinase (MAPK) or homologues thereof in response to various extracellular stimuli. Three distinct groups of MAP kinases have been identified in mammalian cells: 1) extracellular-regulated kinase (Erk1/2 or p44/42), 2) c-Jun N-terminal kinase (JNK) and 3) p38 kinase.

The term “FVIIa-induced activation of the MAPK signaling” is intended to indicate that FVIIa binds to TF in a mammalian cell and thereby induce MAPK signaling.

In a further aspect of the invention, preparation of a humanized antibody comprises testing antibodies in a gene expression analysis assay (see, for example, assay 15) and selecting humanized antibodies which inhibits FVIIa induced up-regulation of genes independently selected from the list comprising Fra-1, Id2, and Cyr61.

Antibodies against TF, which inhibit the activity of TF, may bind different epitopes present on TF and may inhibit the binding of FVIIa as well as or in addition to the binding of FX and/or FXa to human TF. Preferably, antibodies selected by the methods described herein inhibit the binding of FVIIa to human TF and thereby FVIIa-induced intracellular signaling.

In a further aspect, the preparation of a humanized antibody comprises testing antibodies in a human cancer assay (see, e.g., assay 13) and selecting a humanized antibody which inhibits growth or metastasis of human cancers.

In yet a further aspect of the invention, the isolated humanized antibody inhibits FVIIa induced up-regulation of genes independently selected from the list comprising Fra-1, Id2, and Cyr61.

In still a further aspect of the invention, the isolated humanized antibody does not inhibit binding of FX or FXa to human TF.

In another aspect of the invention, the isolated humanized antibody inhibits the intracellular activity of TF.

In a further aspect of the invention, the invention provides a method for the preparation of a humanized antibody comprises testing antibodies in an epitope mapping assay and selecting a humanized antibody that binds one or more selected epitopes on TF. In one aspect, one or more selected epitopes comprises one or more of the residues Trp45, Lys46 and Tyr94. In one aspect, the preferred epitope comprises the residue Trp45. In another aspect the preferred epitope comprises the residue Lys46. In still another aspect the preferred epitope comprises the residue Tyr94.

In a further aspect of the invention, the isolated humanized antibody binds to an epitope within the interface between TF and FVIIa. The residues in TF that are responsible for the interaction between the protease domain of FVIIa and TF determined from the X-ray structure (Banner et al. 1996 Nature, 380: 41-46) are Ser39, Gly43, Trp45, Ser47, Phe50, Arg74, Phe76, Tyr94, Pro92. This interface between the protease domain of FVIIa and TF is characterized as being a complex interface region containing many intermolecular hydrogen bonds allowing many fine contacts between TF and FVIIa to obtain high specificity in binding process of FVIIa.

The present invention also relates to high affinity humanized antibodies to TF. The TF surface containing the contact interface for the protease domain of FVIIa holds a specific topology that is prone to react to create protein-protein interactions, wherein another type of protein-protein interaction is the complex formation between an antibody and a protein ligand.

One aspect of the present invention is high affinity humanized antibodies that immunoreact with the contact interface for the protease domain of FVIIa.

Humanized TF antibodies of the present invention can act as antagonists for TF-mediated induction of coagulation, thus inhibiting the binding of coagulation FVIIa to TF and thereby blocking the production of thrombin and the subsequent deposition of fibrin. The humanized TF antibodies of the invention are particularly useful for administration to humans to treat a variety of conditions involving intravascular coagulation. As such, the inventive humanized TF antibodies may be useful for inhibiting TF activity resulting in, for example, the inhibition of blood coagulation, thrombosis or platelet deposition. Furthermore, humanized TF antibodies according to the present invention, which act to inhibit the cellular functions of TF, for example the signaling function of TF (e.g., as measured by MAPK activity), may be useful in conditions like sepsis, inflammation, atherosclerosis, restenosis, or cancer.

Humanized TF antibodies of the invention may be useful in the prevention, amelioration, and/or treatment of a variety of diseases. Examples of diseases where the use of the humanized antibodies of the invention can be desirable include thrombotic or coagulopathic related diseases or disorders including inflammatory response and chronic thromboembolic diseases or disorders associated with fibrin formation including vascular disorders (such as deep venous thrombosis, arterial thrombosis, post surgical thrombosis, coronary artery bypass graft (CABG), percutaneous transdermal coronary angioplastry (PTCA)), stroke, tumor growth, tumor metastasis, angiogenesis-related disease(s), thrombolysis, atherosclerosis, arteriosclerosis (e.g., arteriosclerosis following angioplastry), and restenosis e.g. restenosis following angioplastry, acute and chronic indications such as inflammation, septic chock, septicemia, hypotension, adult respiratory distress syndrome (ARDS), systemic inflammatory response syndrome (SIRS), disseminated intravascular coagulopathy (DIC), pulmonary embolism, pathological platelet deposition, myocardial infarction, or the prophylactic treatment of mammals with atherosclerotic vessels at risk for thrombosis, venoocclusive disease following peripheral blood progenitor cell (PBPC) transplantation, hemolytic uremic syndrome (H US), thrombotic thrombocytopenic purpura (TTP) and other diseases or disorders. Humanized TF antibodies may be used to prevent the occurrence of thromboembolic complications in identified high risk patients, such as those undergoing surgery or those with congestive heart failure. Humanized TF antibodies may be particularly useful in the treatment of intimal hyperplasia or restenosis due to acute vascular injury. Acute vascular injuries are those which occur rapidly (i.e. over hours to months, in contrast to chronic vascular injuries (e.g. atherosclerosis) which develop over a lifetime. Acute vascular injuries often result from surgical procedures such as vascular reconstruction, wherein the techniques of angioplasty, endarterectomy, atherectomy, vascular graft emplacement or the like are employed. Hyperplasia may also occur as a delayed response in response to, e.g., graft emplacement or organ transplantation. Because humanized TF antibodies are more selective than heparin, binding only TF which has been exposed at sites of injury, and because humanized TF antibodies do not destroy or inhibit other coagulation proteins, the administration of the antibodies of the invention is more effective and less likely to cause bleeding complications than heparin, when used prophylactically (e.g., for the prevention of deep vein thrombosis).

As shown in the Examples that follow, humanized TF antibodies of the invention are able to bind selectively to cell-surface TF and inhibit its functional activity by inhibiting the binding of coagulation FVIIa to TF. Humanized TF antibodies which maintain binding to TF inhibit platelet accumulation at the site of vascular injury by blocking the production of thrombin and the subsequent deposition of fibrin.

Due to the ability of humanized TF antibodies to block thrombin generation and limit platelet deposition at sites of acute vascular injury, humanized TF antibodies which maintain binding to TF thereby inhibiting FVIIa binding can be used to inhibit vascular restenosis.

Compositions comprising humanized TF antibodies are particularly useful in methods of treating patients when formulated into pharmaceutical compositions, where they may be given to individuals suffering from a variety of disease states to treat coagulation-related conditions. Such humanized TF antibodies, capable of binding TF and inhibiting FVIIa binding to TF, may possess a longer plasma half-life and thus a correspondingly longer period of anticoagulant activity when compared to other anticoagulants. Among the medical indications for the subject compositions are those commonly treated with anticoagulants, such as, for example, deep vein thrombosis, pulmonary embolism, stroke, disseminated intravascular coagulation (DIC), fibrin deposition in lungs and kidneys associated with sepsis, antiphospholipid syndrome (APS), atherosclerosis and myocardial infarction. Compositions of the invention can be used to inhibit vascular restenosis as occurs following mechanical vascular injury, such as injury caused by surgery, microsurgery, balloon angioplasty, endarterectomy, reductive atherectomy, stent placement, laser therapy or rotablation, or as occurs secondary to insertion of vascular grafts, stents, bypass grafts or organ transplants. Compositions of the invention can thus be used to inhibit platelet deposition and associated disorders. Thus, the invention provides a method of inhibiting coagulation, vascular restenosis or platelet deposition. For example, the invention provides a method that comprises administering to a patient a composition comprising humanized TF antibodies in an amount sufficient to effectively inhibit coagulation, vascular restenosis, or platelet deposition. The administration of compositions of the invention also find use in the treatment of acute closure of a coronary artery in an individual (e.g. acute myocardial infarction), which comprises administering the humanized TF antibodies in conjunction with tissue plasminogen activator or streptokinase (such a method can accelerate tPA induced thrombolysis). The humanized TF antibodies are given prior to, in conjunction with, or shortly following administration of a thrombolytic agent, such as tissue plasminogen activator.

In another aspect of the invention, human monoclonal antibodies directed against human TF may be produced by immunizing transgenic mice (available through Medarex) carrying parts of the human immune system rather than the mouse system with human TF. Splenocytes from these transgenic mice are used to produce hybridomas that secrete human monoclonal antibodies as described in the art (see, e.g., International Patent Application WO 91/00906, International Patent Application WO 91/10741; International Patent Application WO 92/03918; International Patent Application WO 92/03917; Lonberg, N. et al. 1994 Nature 368: 856-859; Green, L. L. et al. 1994 Nature Genet. 7: 13-21; Morrison, S. L. et al. 1994 Proc. Natl. Acad. Sci. USA 81: 6851-6855; Bruggeman et al. 1993 Year Immunol 7: 33-40; Tuaillon et al. 1993 PNAS 90: 3720-3724; and Bruggeman et al. 1991 Eur J Immunol 21: 1323-1326).

Human monoclonal antibodies directed against human TF may also be produced by phage display techniques. Human antibody libraries can be constructed from immunized persons and displayed on the surface of filamentous phage. High-affinity human single-chain Fv (ScFv) and Fab antibody fragments have in numerous of cases been isolated from such libraries using a panning technique in which the antigen of interest is immobilized on a solid surface, such as microtiter plates or beads (see, e.g., Barbas C. F.,III and Burton, D. R. Trends. Biotechnol. 1996, 14:230-234; Aujame L. et al, Hum. Antibodies 1997, 8:155-68). Phage display of large naïve libraries has also made it possible to isolate human antibodies directly without immunization (see, e.g., DeHaard H. J. et al J. Biol. Chem. 1999, 18218-18230).

The present invention is further illustrated by the following examples which, however, are not to be construed as limiting the scope of protection. The features disclosed in the foregoing description and in the following examples may, both separately and in any combination thereof, be material for realizing the invention in diverse forms thereof.

### EXAMPLES

### Example 1

Preparation of Human Monoclonal Antibodies Immunospecific for Human TF.

Reagents.

Human TF can be isolated from human brain as described by Rao, L. V. M., Thrombosis Research, 51:373-384 1988.

Lipidated recombinant human TF (Dade Innovin, Baxter) can also be used as human thromboplastin reagent. Rat, rabbit, baboon, and pig thromboplastin are prepared from brain tissue. Two volumes of 45° C. 0.9% NaCl are added to the brain tissue, and the tissue is homogenized with a manual glass homogenisator. After 30 min incubation at 45° C. with occasional shaking, the samples are centrifuged 20 min at 2000×g. The precipitate is discarded, and the supernatant is aliquoted and stored at −80° C. until use.

Relipidated TF may be obtained by reconstitution of recombinant human full length TF (American Diagnostica #4500) into phospholipid vesicles (PC/PS 75/25).

Biotinylated human TF is produced as follows: Biotin-NHS (n-succinimido biotin, Sigma H-1759) is dissolved in DMF (dimethylformamid) at a concentration of 1.7 mg/ml. 1 mg/ml of human TF in 0.1 M NaHCO₃ buffer is added 60 μl of the biotin-NHS solution and is allowed to react for 4 hours at room temperature. The reacting solution containing the biotinylated TF is dialyzed against PBS-buffer over night.
FVIIa used is recombinant human FVIIa prepared as described by Thim, L. et al. Biochem 27: 7785-7793,1988.
sTF: Recombinant human soluble TF₁₋₂₀₉ expressed in E. coli and purified essentially as described by Freskgard, P.-O. et al. Protein Sci. 5, 1531-1540 (1996).
S2288: reconstituted in H₂O to 17.24 mg/ml (Chromogenix)
FX: Purified human plasma FX (HFX, Enzyme Research Laboratories Ltd.)
FXa: Purified human plasma FX activated with Russel's Viper Venom (HFXa, Enzyme Research Laboratories Ltd.)
Chromozyme X: Dissolved in H₂O to 1.25 mg/ml. (Boehringer Mannheim)
¹²⁵I-FVIIa is obtained by standard radiolabelling procedures.
FFR-rFVIIa: FVIIa blocked in the active site with D-Phe-L-Phe-L-Arg-chloromethyl ketone. Prepared as described by Sorensen B. B. et al. J. Biol. Chem. 272: 11863-11868, 1997.
   Immunization.

Human TF is emulsified in Freunds Complete Adjuvant. HuMab mice or hybrids thereof (Medarex) are given 40 μg by a subcutaneous injection. After 14 and 28 days, and eventually more times with intervals of 14 days the mice are boosted with a similar injection of 20 μg of TF in Incomplete Freunds Adjuvant. Ten days after the last injection a blood sample is taken and sera are tested for human TF specific antibodies by TF ELISA (Assay 1 and 2).

Fusion.

Mice with positive serum test from assay 1-3 are boosted with 20 μg of human TF by an intravenous injection and sacrificed after three days. The spleen is removed aseptically and dispersed to a single cell suspension.

Fox-myeloma cells are grown in CD Hybridoma medium (Gibco 11279-023).

Fusion of spleen cells and myeloma cells (P3x63 Ag8.653, ATCC CRL-1580), and the Sp2/0 (ATCC CRL-1581) myeloma cell lines for our fusions are done by the PEG-methods (Köhler, G & Milstein C. (1976), European J. Immunology, 6:511-19). Cells are seeded in microtiter plates and incubated at 37° C. Medium is changed three times over the next two weeks. 100 μl of supernatant from hybridoma cells is removed from each well and tested for TF specific antibodies in TF ELISA (Assay 1 and 2).

### Example 2

Screening.

The various assays used in the screening of serum and culture supernatants for selecting specific humanized or human antibodies are described in the following:

Direct TF ELISA Assay (Assay 1):

Nunc immunoplates are coated overnight at 4° C. with 1 μg/ml of human sTF in PBS. Plates are blocked with blocking buffer (TBS with 5 mM CaCl₂ and 2% BSA) and washed in TBS+0.05% Tween 20, and the supernatants from the host cells are added. After incubation at room temperature for 1 hour, plates are washed and anti-human IgG labeled with horseradish peroxidase (HRPO) is added. After another hour of incubation, plates are washed and developed with TMB-substrate (Kem-EN-Tec) as described by the manufactures. Absorbance at 450 nm is measured on an ELISA-reader.

Indirect TF ELISA Assay (Assay 2):

Nunc-immunoplates are coated with 0.5 μg/ml of goat anti-human IgG (Southern Biotechnology Associates, Cat-#2040-1) in PBS and incubated overnight at 4° C. Plates are blocked with blocking buffer (TBS with 5 mM CaCl₂ and 2% BSA) and washed in TBS+5 mM CaCl₂+0.05% Tween 20. Culture supernatants from the host cells are added and the plates incubated for 1 hour at room temperature. After another wash, biotinylated human sTF are added at a concentration of 1 μg/ml, and incubated for 1 hour. After washing, 100 μl of a Streptavidin-HRPO solution is added and incubated for 1 hour. Plates are developed with TMB-substrate as described for assay 1.

FVIIa Competition Assay (Assay 3):

Nunc-immunoplates are incubated with human sTF (conc 5 μg/ml in PBS) over night, 4° C. Plates are washed and blocked in TBS buffer with 5 mM CaCl₂ and 2% BSA. Anti-human TF antibodies are added and plates are incubation for 2 hours. Plates are washed before biotinylated human FVIIa are added (1 μg/ml in TBS buffer with 5 mM CaCl₂ and 2% BSA) and the plates incubated for 1 hour. Plates are washed before addition of HRPO-labeled Streptavidin and incubated for 45 min. Plates are washed again before development with TMB substrate (Kem-EN-Tec) as described by the manufactures.

Inhibition of FVIIa/sTF Amidolytic Activity (Assay 4):

Inhibition of FVIIa-TF catalyzed amidolytic activity by anti-human TF antibodies is tested employing soluble human TF (10 nM), recombinant human FVIIa (10 nM) and increasing concentrations of antibodies (0.0122-50 nM). Varying concentrations of anti-human TF antibodies or FFR-rFVIIa are preincubated with 10 nM sTF and 10 nM FVIIa in BSA buffer (50 mM Hepes, pH 7.4, 100 mM NaCl, 5 mM CaCl₂ and 1 mg/ml BSA) for 60 min at room temperature before addition of substrate S2288 (1.2 mM, Chromogenix). The color development is measured continuously for 30 min at 405 nm. Amidolytic activity is presented as mOD/min. IC₅₀ values for inhibition of FVIIa/TF amidolytic activity by the antibodies may be calculated. The IC₅₀ value for FFR-rFVIIa is 7+/−3 nM in this assay.

Inhibition of FXa Generation (Assay 5).

Lipidated TF (10 pM), FVIIa (100 pM) and anti-TF antibodies or FFR-rFVIIa (0-50 nM) in BSA buffer (see assay 4) are incubated 60 min at room temperature before FX (50 nM) is added. The reaction is stopped after another 10 min by addition of ½ volume stopping buffer (50 mM Hepes, pH 7.4, 100 mM NaCl, 20 mM EDTA). The amount of FXa generated is determined by adding substrate S2765 (0.6 mM, Chromogenix, and measuring absorbance at 405 nm continuously for 10 min. IC₅₀ values for antibody inhibition of FVIIa/lipidated TF-mediated activation of FX may be calculated. The IC₅₀ value for FFR-rFVIIa is 51+/−26 pM in this assay.

Biosensor Assay (Assay 6):

antibodies are tested on the Biacore instrument by passing a standard solution of anti-human TF antibody over a chip with immobilized antibody to human IgG. This is followed by different concentrations of sTF in 10 mM hepes pH 7.4 containing 150 mM NaCl, 10 mM CaCl₂ and 0.0003% polysorbate 20. K_{d} values are calculated from the sensorgrams using the integrated Biacore evaluation software.

TF-Dependent Clotting Assay (Assay 7):

The assay is carried out on an ACL300 Research clotting apparatus (ILS Laboratories). Dilutions of anti-human TF antibodies in 50 mM imidazole, pH 7.4, 100 mM NaCl, 0.1% BSA are mixed with 25 mM CaCl₂ in the ratio of 2 to 5 and added to sample cups in the clotting apparatus. Thromboplastin from human, rat, rabbit, baboon, or pig diluted with the imidazole buffer to give clotting time of approximately 30 sec in samples without antibody is placed in reagent reservoir 2, and human, rat, rabbit, baboon, or pig plasma, in reagent reservoir 3. During the analysis 70 μl of the antibody and CaCl₂ mixture is transferred to 25 μl thromboplastin reagent and preincubated 900 sec before addition of 60 μl plasma and measuring of the clotting time. Maximal clotting time is set to 400 sec. A dilution of the thromboplastin is used as standard curve for converting clotting times into TF activity relative to the control without anti-TF antibodies or FFR-rFVIIa added. The IC₅₀ value for FFR-rFVIIa is 4.4+/−0.4 pM in this assay.

Inhibition of FVIIa/Cell Surface TF Catalyzed Activation of FX by Anti TF Antibodies (Assay 8):

Monolayers of cells expressing human TF, e.g. human lung fibroblasts WI-38 (ATTC No. CCL-75), human bladder carcinoma cell line J82 (ATTC No. HTB-1), human keratinocyte cell line CCD 1102KerTr (ATCC no. CRL-2310), human glioblastoma cell line U87, or human breast cancer cell line MDA-MB231, are employed as TF source in FVIIa/TF catalyzed activation of FX. Confluent cell monolayers in a 24-, 48- or 96-well plate are washed one time in buffer A (10 mM Hepes, pH 7.45, 150 mM NaCl, 4 mM KCl, and 11 mM glucose) and one time in buffer B (buffer A supplemented with 1 mg/ml BSA and 5 mM Ca²⁺). FVIIa (1 nM), FX (135 nM) and varying concentrations of antibody (or FFR-rFVIIa) in buffer B are simultaneously added to the cells. Alternatively the cells are preincubated 15 min with anti-TF antibodies or FFR-rFVIIa before addition of rFVIIa and FX. FXa formation is allowed for 15 min at 37° C. 50-μl aliquots are removed from each well and added to 50 μl stopping buffer (Buffer A supplemented with 10 mM EDTA and 1 mg/ml BSA). The amount of FXa generated is determined by transferring 50 μl of the above mixture to a microtiter plate well and adding 25 μl Chromozym X (final concentration 0.6 mM) to the wells. The absorbance at 405 nm is measured continuously and the initial rates of color development are converted to FXa concentrations using a FXa standard curve. The IC₅₀ value for FFR-rFVIIa is 1.5 nM in this assay.

Inhibition of ¹²⁵I-FVIIa Binding to Cell Surface TF by Antibody (Assay 9):

Binding studies are employed using cells expressing human TF, e.g. human lung fibroblasts WI-38 (ATTC No. CCL-75), human bladder carcinoma cell line J82 (ATTC No. HTB-1), human keratinocyte cell line CCD 1102KerTr (ATCC no. CRL-2310), human glioblastoma cell line U87, or human breast cancer cell line MDA-MB231. Confluent monolayers in 24-well tissue culture plates are washed once with buffer A (see assay 8) and once with buffer B (see assay 8). The monolayers are preincubated 2 min with 100 μl cold buffer B. Varying concentrations of antibodies (or FFR-rFVIIa) and radiolabelled FVIIa (0.5 nM ¹²⁵I-FVIIa) are simultaneously added to the cells (final volume 200 μl). The plates are incubated for 2 hours at 4° C. At the end of the incubation, the unbound material is removed, the cells are washed 4 times with ice-cold buffer B and lysed with 300 μl lysis buffer (200 mM NaOH, 1% SDS and 10 mM EDTA). Radioactivity is measured in a gamma counter (Cobra, Packard Instruments). The binding data are analyzed and curve fitted using GraFit4 (Erithacus Software, Ltd., (U.K.). The IC₅₀ value for FFR-rFVIIa is 4 nM in this assay.

Inhibition of FVIIa/TF-Induced p44/42 MAPK Activation by Anti TF Antibody (Assay 10):

The amount of phosphorylated p44/42 MAPK and/or Akt, and/or p90RSK is determined by quantitative detection of chemiluminescence (Fujifilm LAS-1000) from western blot analysis. Cells expressing human TF, e.g. CCD1102KerTr, NHEK P166, human glioblastoma cell line U87, or human breast cancer cell line MDA-MB231, are cultured in medium with 0-0.1% FCS for 24 or 48 hours prior to the experiment to make cells quiescent. At the day of the experiment the cells must be 70-80% confluent. The experiment is performed by preincubating the cells with excess antibody or FFR-rFVIIa in medium without serum for 30 min at 37° C. before addition of 10-100 nM FVIIa and incubating for 10 min. As a positive control of cell signaling, cells are treated with 10% FCS for 10 minutes. Cells are washed 2 times in ice-cold PBS before cells are lysed in lysis buffer (20 mM Tris, 0.1% Triton X-100, 1 mM EDTA, 1 mM EGTA, 50 mM sodium-fluoride, 10 mM sodium β-glycerophosphate, 5 mM sodium pyrophosphate, 150 mM NaCl, pH 7.5 containing 0.1 mM 4-(2-aminoethyl)benzene-sulfonyl fluoride (AEBSF) and 1 mM benzamidine. Added just before use: 1 mM sodium orthovanadate, 5 μg/ml leupeptin, 10 μg/ml aprotinin). Lysates were mixed with SDS-sample buffer and loaded on a SDS-polyacrylamide gel. A standard biotinylated protein marker is loaded on each gel. Proteins separated on the SDS-polyacrylamide gel were transferred to nitrocellulose by electroblotting, and the kinases p44/42 MAPK, Akt and p90RSK were visualized by immunoblotting with phosphospecific antibodies, and chemiluminiscence is quanitiated by Fujifilm LAS1000.

Epitope Mapping Assay (Assay 11):

Preparation of Soluble TF (sTF) Variants.

sTF variants (I22C, W45C, K46C, Y94C, F140C, W158C, K201C) are constructed using inverse PCR (QuikChange, Stratagene, La Jolla, Calif., USA) using the wild type plasmid (Freskgard et al. Protein Sci. 5, 1531-1540, 1996) as template. The wild type and variants are expressed and purified in E. coli as described elsewhere (Freskgard et al., Protein Sci. 5, 1531-1540, 1996) with some modifications. The cell lysis is performed by the X-press (Biox, Sweden) technique in 10 mM Tris-HCl buffer, pH 7.5 and thereafter resuspended in the same buffer with the addition of 1 mg of DNAse. The solution is centrifuged at 11000×g for 20 min at 4° C., and the inclusion bodies are denatured in 75 ml of 6 M GuHCl, 0.5 M NaCl, 20 mM Tris-HCl, pH 8.0. Refolding is achieved after 1-hour incubation at room temperature by dropwise diluting the denatured protein into a 1 L solution containing 50 mM Tris-HCl, 0.25 M NaCl, pH 8.0 with gentle stirring for approximately 2 hours. Purification is performed using O-Sepharose Fast Flow (Pharmacia, Uppsala, Sweden) and FVIIa affinity chromatography as described by Freskgård et al. (1996). The homogeneity of the protein is verified by SDS-PAGE. The concentration is measured at A_{280 nm} and determined using a calculated extinction coefficient of 37440 M⁻¹cm⁻¹ (Gill and von Hippel, 1989).

MaxiSorp plates (Nunc-Immuno) are coated with wild type sTF and the variants (10 μg/ml) in TBS and blocked with blocking buffer (TBS with 0.1% Tween 20 and 0.5% BSA). The plates are washed with washing buffer (TBS and 0.1% Tween 20). The anti-human TF antibodies are applied at a concentration of 1 ng/ml in blocking buffer and incubated for one hour. The plates are then washed (6×) using the washing buffer. The antibody binding is subsequently detected using an HRP-labeled anti-human IgG (Helica Biosystems, Inc) at a 1:2000 dilution in blocking buffer using the TMBₚₗᵤₛ-substrate (Kem Tech Cat. 4390A). The final ELISA signal (OD₄₅₀₋₆₂₀) is used as a measure of the binding of each antibody to all sTF variants.

Thromboelastography (Assay 12)

Human thromboplastin (e.g. Innovin, Dade Behring, final dilution 50,000×) is mixed with CaCl₂ (final concentration 16.7 mM) and anti-TF antibodies and incubated 15 min at room temperature. Citrate-stabilized human whole blood (280 μl) is added to RoTEG sample cups (Pentapharm) and preheated 5 min at 37° C., before addition of 40 μl thromboplastin/CaCl₂/anti-TF antibody mixture. Thromboelastography is followed for one hour in a RoTEG apparatus (Pentapharm). Velocity profiles are obtained from the thrombograms using CoagPro Software™ (MedScience, Århus, Denmark).

### Example 3

Human Cancer Assay. Investigating the Effects of Treatment with Human Anti-TF Antibodies on Growth and Metastasis of Human Cancers in Mouse Models (Assay 13)

Treatment:

Human anti-TF antibodies given by bolus injection i.v.; 10 mg/kg=0.1 mg/10 g; Injection-volume is 0.1 ml per 10 g mouse of either of three treatment solutions:
A. Vehicle control
B. 1 mg/ml Human FFR-rFVIIa
C. 1 mg/ml anti-TF antibodies
   Description of Models:
   I. Primary Growth and Liver Metastasis of Colon Cancer

Healthy female athymic mice (nu/nu) aged 7-8 weeks are used. To destroy the residual immunoresistance of the nude mice to the human cell implantation, the mice are routinely irradiated at 5 Gy 2 days before human tumor grafting (Vogel et al., 1997). Mice are challenged by tumor grafting of LS174T human colon carcinoma cells (ATCC CCL 188) cultured in RPMI 1640 with 15% fetal calf serum (FCS) as described (Li et al., Human Gene Therapy 10: 3045-3053, 1999). In brief, the cells are harvested with trypsin-EDTA, washed twice, and resuspended in serum free RPMI supplemented with sodium-heparinate solution (1 U/ml). A small left subcostal incision is then carried out in mice under anesthesia and 3 3 106 LS174T cells in 50 m lof phosphate-buffer ed saline (PBS) are injected into the spleen. After 3 to 5 min, the spleen vessels are ligated and the spleen is surgically removed. This procedure will lead to a stable incidence of liver metastasis (more than 95%). The treatment with anti-TF antibodies will be initiated immediately after implantation and will last for the remaining study period. On days 15 and 30 after tumor cell inoculation mice are sacrificed, the livers ae removed and weighed, and the number of visible tumor nodules on the liver surfaces are counted. Liver samples are fixed overnight in AFA (5% acetic acid, 75% ethyl alcohol, 2% formalin, 18% water), transferred to 100% ethanol, and embedded in paraffin, and 5-m m sections are prepared for histological quantification of metastatic nodules, for immunohistochemistry and apoptosis quantification.

Study I-1:

Aim: To examine the effect on macroscopical growth and liver metastasis of LS174T colon tumors in nude mice of anti-TF antibodies given bolus injection i.v.; 10 mg/kg.
Mice: 60 homozygotous nu/nu 6 weeks old NMRI males.
Groups Mice are randomly allocated in four groups of 15 and treated with solutions A, B, or C.
Termination: At a weight loss of >20% or other objective signs of severe toxicity the animal is terminated.
Parameters: Tumor size in two orthogonal diameters is recorded daily during the growth phase. The body weight is recorded initially and 2-3 times per week. Postmortem determination of metastasis formation in the liver.
   II. Primary Growth and Lung Metastasis of Mammary Cancer

Human breast carcinoma cells MDA-MB-231 (ATCC HTB26) are cultured in Dulbecco's modified Eagle's medium (DMEM) supplemented with 10% fetal calf serum (FCS). MDA-MB-231 cells (3 3 106) are injected subcutaneously in nude mice (7- to 8-week-old female mice). Primary tumor growth and metastasis is evaluated as described previously (Li et al., Human Gene Therapy 12: 515-526, 2001).

Study II-1:

Aim: To examine the effect on macroscopical growth and lung metastasis of MDA-MB-231 mammary tumors in nude mice of anti-TF antibodies given bolus injection i.v.; 10 mg/kg.
Mice: 60 homozygotous nu/nu 6 weeks old NMRI males.
Groups: Mice are randomly allocated in four groups of 15 and treated with solutions A, B, or C.
Termination: At a weight loss of >20% or other objective signs of severe toxicity the animal is terminated.
Parameters: Tumor size in two orthogonal diameters is recorded daily during the growth phase. The body weight is recorded initially and 2-3 times per week. Postmortem determination of metastasis formation in the lung.
   III. Primary Growth of Glioma Tumor Xenografts

The tumor cell line MG U373 is a human glioblastoma multiforme cell line, with high angiogenic activity, high vascular density and fast growth in nude mice. Tumors are inoculated in the flanks, following standard procedures (see enclosed protocols for experimental plan). The mice are observed twice daily for signs of toxicity and the tumors are measured daily in two perpendicular diameters.

Tumors are transplanted to the flanks of nu/nu homozygotous nude mice of NMRI background. The mice are 7-week-old males obtained from M&B (Ry, Denmark). Animals are kept in a gnotobiotic environment and they receives sterile food pellets and drinking water ad libitum.

Three different studies is conducted with the glioma tumor model:

Study III-1:

Aim: To examine the effect on macroscopical growth of U373 tumors in nude mice of anti-TF antibodies given bolus injection i.v.; 10 mg/kg.
Mice: 60 homozygotous nu/nu 6 weeks old NMRI males.
Groups: Mice are randomly allocated in four groups of 15 and treated with solutions A, B, or C.
Termination: At a weight loss of >20% or other objective signs of severe toxicity the animal is terminated.
Parameters: Tumor size in two orthogonal diameters is recorded daily during the growth phase. The body weight is recorded initially and 2-3 times per week.
   Study III-2:
Aim: To examine the effect on macroscopic growth of U373 tumors in nude mice of anti-TF antibodies given bolus injection i.v.; 10 mg/kg. after pretherapeutic tumor growth has been established.
Mice: 60 homozygotous nu/nu 6 weeks old NMRI males.
Groups: Mice are randomly allocated in four groups of 15 and treated with solutions A, B, or C. Treatment starts when 6 consecutive (daily) measurements show Gompertzian growth. This corresponds to 100-200 mm³
Termination: Treatment lasts until the tumors have grown beyond the maximal size of approximately 1.0 cm³, i.e. no tumor diameter larger than 15 mm or until Gompertzian regrowth has been established by 6 consecutive measurements. At time of termination tumors from each group are excised for histological and immunochemical evaluation. At a weight loss of >20% or other objective signs of severe toxicity the animal is terminated
Parameters: Tumor size in two orthogonal diameters is recorded daily during the growth phase. The body weight is recorded initially and 2 times per week.
   Study III-3:
Aim: To examine the effect of anti-TF antibodies on growth of intracranial U373 tumors in nude mice.
Mice: 60 homozygotous nu/nu 6 weeks old NMRI males.
Tumor: U373 implanted orthotopically in the right hemisphere following standard procedures.
Groups: Mice are randomly allocated in four groups of 15 and treated with solutions A, B, or C.
Termination: Mice with signs of chronic neurological impairment are euthanized.
Data: Survival (i.e. time to neurological impairment) is quantified by Kaplan-Meyer statistics.

### Example 4

### Assay 14

In mouse wherein the TF gene is knocked out and human TF gene is inserted (mTF-KO/hTF-KI mice) a 0.5 ml matrigel plug will be located subcutaneously under the abdominal skin. In the matrigel b-FGF (5 ng) will be incorporated and one week later the formation of new patent vessels in the gel will be quantitated by measuring the content of haemoglobin (angiogenesis). The inhibitory capacity (% inhibition of the haemoglobin content) of the human anti-TF antibodies can be evaluated after single or repeated parenteral administrations of the proteins.

### Example 5

### Assay 15

Gene Expression Analysis Assay for Discriminating Antibodies, that Prevents FVIIa Binding to TF and Antibodies, that Prevents FX Binding to TF.

In cDNA microarray analyses a specific up-regulation of three genes in BHK-TF cells treated with FVIIa has been observed. These include: Fra-1, a gene coding for Fos related antigen 1, Id2, a gene encoding a member of the helix-loop-helix class of proteins, and Cyr61 encoding an extracellular matrix signaling protein. The following assay is designed to screen for anti-TF antibodies which prevents FVIIa induced up-regulation of Fra-1, Id2 or Cyr61.

Cell Culture.

Reagents are purchased from GIBCO-BRL Life Technologies unless otherwise noted.

BHK-TF cells, created as described by Poulsen L. K. et al., J. Biol. Chem. 273, 6228-6232, 1998, are grown in Dulbecco's modified Eagle's medium containing 10% FCS, 100 IU/ml penicillin, and 100 μg/ml streptomycin to obtain 95-100% confluence, washed and grown for additional 16-18 hs in medium without FCS. The cells are again washed and exposed to FCS-free medium containing 100 nM FVIIa.

For cloning of fragments for Northern blot analyses the cells are treated as follows. BHK-TF cells are grown in Dulbecco's modified Eagle's medium containing 10% FCS, 100 IU/ml penicillin, and 100 μg/ml streptomycin to obtain 95-100% confluence, washed and grown for additional 16-18 hs in medium without FCS. The cells are again washed and exposed to FCS-free medium containing 100 nM FVIIa for 1 h. CRL2091 cells (ATCC) are grown in Iscove's modified Dulbecco's medium containing 10% FBS, 100 U/ml penicillin, and 100 μg/ml streptomycin to 95-100% confluence. Subsequently, the cells are serum-starved for 16-18 hs and treated with FBS-free medium containing 100 nM FVIIa for 6 hs. Murine 3T3-L1 cells (ATCC) are maintained in Dulbecco's modified Eagle's medium supplemented with 10% fetal bovine serum, 100 U/ml penicillin, and 100 μg/ml streptomycin. Cells are grown to confluence and induced to with media containing 1 μM dexamethasone (Sigma), 10 μg/ml human insulin (Novo Nordisk A/S), and 1 μM BRL49653 (Novo Nordisk A/S) for 1 h.

Cloning of Fragments for Northern Blot Analyses.

Fra-1 is cloned by reverse transcription PCR from RNA isolated from 3T3-L1 cells treated for 1 h with dexamethasone, insulin, and BRL49653 using the superscript 11 kit (Life Technologies) according to the manufacturer's instructions. Id2 and Cyr61 are cloned by reverse transcription PCR from RNA isolated from BHK-TF cells treated for 1 h with FVIIa and from CRL2091 cells treated for 6 hours with FVIIa, respectively. The upstream and downstream primers are: 5′-GCGGCCGCCATGTACCGAGACTACGGGGAACCG-3′ and 5′-GCGGCCGCTCACAAAGCCAGGAGTGTAGG-3′ for Fra-1,5′-CAGCATGAAAGCCTTCAGTC-3′ and 5′-CTCTGGTGATGCAGGCTGAC-3′ for Id2,5′-CGTCACCCTTCTCCACTTGA-3′ and 5′-CTTGGTCTTGCTGCATTTCT-3′ for Cyr61. Parameters for PCR are one cycle of denaturing at 94° C. for 10 s, annealing at 65° C. for 15 s, and extension at 72° C. for 1.5 min, one cycle of denaturing at 94° C. for 10 s, annealing at 64° C. for 15 s, and extension at 72° C. for 1.5 min, one cycle of denaturing at 94° C. for 10 s, annealing at 63° C. for 15 s, and extension at 72° C. for 1.5 min, one cycle of denaturing at 94° C. for 10 s, annealing at 62° C. for 15 s, and extension at 72° C. for 1.5 min, one cycle of denaturing at 94° C. for 10 s, annealing at 61° C. for 15 s, and extension at 72° C. for 1.5 min, one cycle of denaturing at 94° C. for 10 s, annealing at 60° C. for 15 s, and extension at 72° C. for 1.5 min, 40 cycles of denaturing at 94° C. for 10 s, annealing at 55° C. for 15 s, and extension at 72° C. for 1.5 min. All fragments are cloned into into TOPO 2.1 (Invitrogen) and sequenced using a Megabase sequencer.

Northern Blot Analysis.

Total RNA are isolated from BHK-TF cells incubated with FVIIa, FX, ASIS, 1F44A1 or TF8-5G9 using TriZol following the instructions of the vendor. 20 μg of RNA are size-fractionated in a denaturing gel containing 1% agarose, 20 mM MOPS, 5 mM NaOAc, 6% formaldehyde, and 1 mM EDTA, transferred to a Hybond N⁺ membrane (Amersham) by capillary blotting and immobilized by UV crosslinking. cDNA encoding Fra-1, Id2 or Cyr61 are labeled with the Prime It kit (Stratagene) using [α-³²P] dATP (Amersham) and hybridized using Express Hyb (Clontech) following the manufacturer's instructions and results are visualized by autoradiography.

### Example 6

### Assay 16

MAPK Assay Via the Elk1 Transcription Factor/Luciferase Reporter (PathDetect)

HeLa cells are seeded to 40% confluence in a T-80 flask one day prior to transfection. Cells are transfected with 150 ng pFA-Elk1 (Stratagene), 3 μg pFR-Luc (Stratagene), 3 μg human TF/pcDNA3 and 3 μg mouse Protease Activated Receptor 2/pcDNA3,1+using 36 μl FuGene (Roche) as described in the manual. The following day the cells are detached by Versene™ (Invitrogen) and seeded in black 96 well view plates (Packard) at a cell density of 20.000 cells per well. After the cells had reattached to the plate, the medium is replaced with 160 μl per well serum-free Dulbeccos Modified Eagle Medium (Invitrogen) and incubated for 16 hours.

Cells are preincubated for 1 hour with either 20 μl serum-free medium (control), 20 μl 2.5 μM FFR-rFVIIa (control), 20 μl 2.5 μM anti TF antibody B or 20 μl 2.5 μM anti TF antibody A. 20 μl 0.5 μM FVIIa is added to half of the wells and medium to the other half. Following 4 hours of incubation the cells are subjected to the Luciferase gene assay. LucLite (Packard) reagent is added to the cells as described by the manufacturer. Luciferase expression levels are read on a TopCount Microplate Scintillation (Packard).

### Example 7

Preparation of Humanized Antibodies Immunospecific for Human TF.

Human monoclonal antibodies are made as described in example 1. These antibodies have relatively low affinity to tissue factor compared to murine monoclonal antibodies made against the same epitope. The hyper variable regions of the murine monoclonal antibodies have been sequenced and transferred to a human monoclonal antibody that reacts with the same epitope on tissue factor as well as to a “naïve” human monoclonal antibody framework. Affinities have been compared between the two antibody frameworks.

Antibody Generation.

Transgenic mice expressing human antibodies and normal mice (e.g. Balb/C or RBF) are three times immunized with the antigen, here human TF. 10 days after the last immunization the antibody titer were determined and the highest responder of the transgenic mice and the normal mice were selected for fusions. Spleen cells of these mice were isolated and fused to the mouse myeloma cell line AG8.X63 in the case of the transgenic mice and FOX in the case of the normal mice. After 14 days of selection clones were screened for binding towards human TF. Afterwards the affinity of positive clones is measured by BIAcore.

mRNA Extraction and cDNA Synthesis.

mRNA from clones of the highest affinity mouse and human antibodies is isolated and used as template for cDNA synthesis by RT-PCR amplification of the hypervariable regions according to standard procedures.

Amplification of Murine TF Antibody Variable Regions.

TF antibody variable heavy chains are amplified from cDNA using a degenerated primer mix of: 5′-ACTAGTTTTGGCTGAGGAGACGGTGACCGTGG-3′,5′-ACTAGTTTTGGCTGAGGAGACTGTGAGAGTGG-3′,5′-ACTAGTTTTGGCTGCAGAGACAGTGACCAGAG-3′,5′-ACTAGTTTTGGCTGAGGAGACGGTGACTGAGG-3′ and a universal primer mix present in SMART RACE kit supplied by Clontech (kat.# K1811-1).

TF antibody variable kappa-light chains are amplified from cDNA using 5′-TCATCAACACTCATTCCTGTTGAAGCTCTTGA-3′ and a universal primer mix present in SMART RACE kit supplied by Clontech (kat.# K1811-1).

Amplification of Human TF Antibody Variable Regions.

HuTF antibody variable heavy chains are amplified from cDNA of using primer 5′-GTGCCAGGGGGAAGACCGATGGG-3′ and a universal primer mix present in SMART RACE kit supplied by Clontech (kat.# K1811-1).

HuTF antibody variable kappa-light chains are amplified from cDNA using primer 5′-GCAGGCACACAACAGAGGCAGTTCCAGATTTC-3′ and a universal primer mix present in SMART RACE kit supplied by Clontech (kat.# K1811-1).

Cloning of TF Antibodies into E. coli

The PCR product of the TF antibody variable chains are TOPO-cloned according to manufactures protocol (Invitrogen kat.#45-0030) and transformed in competent E. coli cells. DNA is extracted from transformants and the variable regions are characterized by sequencing.

Genetic Modification of HuTF Antibody Hyper Variable Regions.

The CDRs are characterized by sequencing from which conserved consensus motifs can be derived. Based on the sequence data obtained from HuTF and murine TF antibody variable regions new CDRs are engineered and introduced into the HuTF antibody framework. The new CDRs are created in the variable regions by extension PCR and the engineered variable regions are introduced into IgG expression vectors pIESRg1, pIESRg1f, and pIESRg4 (provided by Medarex) in the following manner: Variable light chains introduced into HindIII/BsiWi site and variable heavy chains introduced into NotI/NheI site of the vectors.

Expression of Recombinant TF Antibodies.

The resultant plasmids are analyzed by sequencing and transfected into mammalian cells according to standard procedures. Antibodies are purified from the supernatant by protein A and affinities between those recombinant generated antibodies are compared.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An isolated humanized antibody that immunoreacts with an epitope present on human TF and inhibits the binding of human coagulation factor Vila to human TF.

2. The isolated humanized antibody of claim 1, wherein the CDR amino acid sequences of the humanized antibody are derived from a parent monoclonal antibody.

3. The isolated humanized antibody of claim 2, wherein the parent monoclonal antibody is a mouse monoclonal antibody.

4. The isolated humanized antibody of claim 1, wherein the humanized antibody is a Fab fragment.

5. The isolated humanized antibody of claim 1, wherein the humanized antibody is a F(ab)₂ fragment.

6. The isolated humanized antibody of claim 1, wherein the humanized antibody is a F(ab′)₂ fragment.

7. The isolated humanized antibody of claim 1, wherein the humanized antibody is a single chain Fv fragment.

8. The isolated humanized antibody of claim 1, wherein the humanized antibody has a K_{d} for binding to human TF of from about 10⁻¹⁵ to about 10⁻⁸ M.

9. The isolated humanized antibody of claim 8, wherein the humanized antibody has a K_{d} for binding to human TF of from about 10⁻¹⁵ to about 10⁻¹⁰ M.

10. The isolated humanized antibody of claim 9, wherein the humanized antibody has a K_{d} for binding to human TF of from about 10⁻¹⁵ to about 10⁻¹² M.

11. The isolated humanized antibody of claim 1, wherein the human framework amino acid sequences of the humanized antibody are derived from a human antibody that immunoreacts with a second epitope present on human TF.

12. The isolated humanized antibody of claim 11, wherein the second epitope comprises an amino acid residue at a particular position of human TF that also is comprised within the epitope.

13. The isolated humanized antibody of claim 12, wherein the amino acid residue at a particular position of human TF is selected from the group consisting of Trp45, Lys46, and Tyr94.

14. The isolated humanized antibody of claim 11, wherein the epitope or second epitope comprises an amino acid residue selected from the group consisting of Trp45, Lys46, and Tyr94.

15. A pharmaceutically acceptable composition comprising a therapeutically effective amount of the isolated humanized antibody of claim 1.

16. A pharmaceutically acceptable composition comprising a therapeutically effective amount of the isolated humanized antibody of claim 12.

17. A method for treating a FVIIa/TF related disorder in a human comprising administering a therapeutically effective amount of a humanized antibody that immunoreacts with an epitope on human TF and inhibits the binding of human coagulation factor VIIa to human TF.

18. A method for preparing a humanized antibody comprising preparing humanized antibodies against human TF and (i) testing the antibodies in a FVIIa/TF amidolytic assay and selecting a humanized antibody that inhibits TF-induced FVIIa amidolytic activity with an IC₅₀ value that is less than the IC₅₀ value of FFR-rFVIIa+about 100 nM (using 10 nM FVIIa in the assay), (ii) testing the antibodies in a FVIIa competition assay and selecting a humanized antibody that compete with FVIIa binding, or (iii) testing the antibodies in a FVIIa signaling assay and selecting a humanized antibody that inhibits FVIIa-induced intracellular signaling.

19. The method of claim 21, wherein the method comprises testing antibodies in a FVIIa/TF amidolytic assay and selecting a humanized antibody that inhibits TF-induced FVIIa amidolytic activity with an IC₅₀ value less than the IC₅₀ value of FFR-rFVIIa+about 100 nM (using 10 nM FVIIa in the assay).

20. The method of claim 18, wherein the method comprises testing antibodies in a FVIIa competition assay and selecting a humanized antibody which competes with FVIIa binding.

21. The method of claim 18, wherein the method comprises testing antibodies in a FVIIa signaling assay and selecting a humanized antibody that inhibits FVIIa-induced intracellular signaling.

22. A method of preparing a humanized antibody comprising:
(a) preparing immortal cells that secrete humanized antibodies;
(b) isolating culture medium from the immortal cells comprising humanized antibodies,
(c) testing the antibodies in (i) an indirect TF ELISA assay comprising TF in solution and selecting a humanized antibody therefrom that detectably immunoreacts with human TF in solution; (ii) a FVIIa competition assay and selecting a humanized antibody therefrom that detectably competes with FVIIa binding; (iii) a FVIIa/TF amidolytic assay and selecting a humanized antibody therefrom that inhibits TF-induced FVIIa amidolytic activity with an IC₅₀ value that is less than the IC₅₀ value of FFR-rFVIIa+about 100 nM (using 10 nm FVIIa in the assay); (iv) a FXa generation assay and selecting a humanized antibody therefrom that inhibits FXa generation with an IC₅₀ value that is less than the IC₅₀ value of FFR-rFVIIa+about 100 nM (using 0.1 nM FVIIa in the assay); (v) a TF-induced clot assay and selecting a humanized antibody therefrom that inhibits clot formation with an IC₅₀ value that is less than the IC₅₀ value of FFR-rFVIIa+about 1 nM; or (vi) any combination of (i)-(v) and selecting a humanized antibody that meets the criteria of the selected tests,
(d) cultivating a cell that produces the humanized antibody, and
(e) isolating the humanized antibody.

23. The method of claim 22, wherein the method comprises testing the antibodies in a direct TF ELISA assay comprising immobilized TF and selecting humanized antibodies that immunoreact with immobilized human TF.

24. The method of claim 23, wherein the method further comprises testing antibodies in a FXa generation assay on a TF expressing cell and selecting a humanized antibody that inhibits FXa generation on TF expressing cell with an IC₅₀ value less than the IC₅₀ value of FFR-rFVIIa+about 500 nM (using 1 nM FVIIa in the assay).

25. The method of claim 22, wherein the method comprises testing the antibodies in a whole cell TF binding assay and selecting a humanized antibody that competes with FVIIa binding to human TF expressed on the surface of the whole cells.

26. The method of claim 22, wherein the method comprises testing the antibodies in a biosensor assay and selecting a humanized antibody with a K_{d} for binding to human TF of less than about 100 nM.

27. The method of claim 22, wherein the method comprises testing the antibodies in a FVIIa signaling assay and selecting a humanized antibody that inhibits FVIIa-induced intracellular signaling.

28. The method of claim 22, wherein the method comprises testing the antibodies in an epitope mapping assay and selecting a humanized antibody that immunoreacts with one or more selected epitopes on TF.

29. The method of claim 28, wherein the preferred epitope comprises an amino acid residue selected from the group consisting of Trp45, Lys46, and Tyr94.

30. A cell that produces a humanized antibody that immunoreacts with an epitope on human TF and inhibits the binding of human coagulation factor VIIa to human TF.

31. The cell of claim 30, wherein the cell is a mammalian cell.

32. The cell of claim 30, wherein the cell is a selected from the group consisting of CHO, BHK, HEK293, P3×63-Ag8, P3×63-AG8.653, PERC6, NSO, YB2/0, P3/NS1-Ag4-1 (NS-1), Sp2/0-Ag14 and S194/5.XXO.Bu.1.

33. An isolated humanized antibody that immunoreacts with an epitope on a protein, wherein the human framework amino acid sequences of the humanized antibody are derived from a human antibody that immunoreacts with a second epitope on the protein and the second epitope comprises an amino acid residue at a particular position of the protein that also is comprised within the epitope.


SEQUENCE LISTING (13 sequences)

SEQ ID NO: 1 - DNA, 33 bp, Artificial
  Synthetic
gcggccgccatgtaccgagactacggggaaccg

SEQ ID NO: 2 - DNA, 29 bp, Artificial
  Synthetic
gcggccgctcacaaagccaggagtgtagg

SEQ ID NO: 3 - DNA, 20 bp, Artificial
  Synthetic
cagcatgaaagccttcagtc

SEQ ID NO: 4 - DNA, 20 bp, Artificial
  Synthetic
ctctggtgatgcaggctgac

SEQ ID NO: 5 - DNA, 20 bp, Artificial
  Synthetic
cgtcacccttctccacttga

SEQ ID NO: 6 - DNA, 20 bp, Artificial
  Synthetic
cttggtcttgctgcatttct

SEQ ID NO: 7 - DNA, 32 bp, Artificial
  Synthetic
actagttttggctgaggagacggtgaccgtgg

SEQ ID NO: 8 - DNA, 32 bp, Artificial
  Synthetic
actagttttggctgaggagactgtgagagtgg

SEQ ID NO: 9 - DNA, 32 bp, Artificial
  Synthetic
actagttttggctgcagagacagtgaccagag

SEQ ID NO: 10 - DNA, 32 bp, Artificial
  Synthetic
actagttttggctgaggagacggtgactgagg

SEQ ID NO: 11 - DNA, 32 bp, Artificial
  Synthetic
tcatcaacactcattcctgttgaagctcttga

SEQ ID NO: 12 - DNA, 23 bp, Artificial
  Synthetic
gtgccagggggaagaccgatggg

SEQ ID NO: 13 - DNA, 32 bp, Artificial
  Synthetic
gcaggcacacaacagaggcagttccagatttc